SEQUENCE LISTING (32 sequences)

SEQ ID NO: 1 - DNA, 971 bp, homo sapiens
gtccgggtgacgtctcccgagggcgtcggcagggtcggcggcgtcggcagcagtgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccatcttgattcaaaacatctatcgtaatccccaaaacagtgcacagacggctgacggctcacactgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttttaaaaaacaacaaaaaaaaaaacatacaaagatgggtttctgaataaaatttgtagtgataacagtaaaaaaaaaaaaaaaaaaa

SEQ ID NO: 2 - DNA, 1038 bp, homo sapiens
gtccgggtgacgtctcccgagggcgtcggcagggtcggcggcgtcggcagcagtgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccatcttgattcaaaacatctatcgtaatccccaaaacagtgcacagacggctgacggctcacactaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttttaaaaaacaacaaaaaaaaaaacatacaaagatgggtttctgaataaaatttgtagtgataacagtaaaaaaaaaaaaaaaaaaa

SEQ ID NO: 3 - DNA, 971 bp, homo sapiens
gtccgggtgacgtctcccgagggcgtcggcagggtcggcggcgtcggcagcagtgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttttaaaaaacaacaaaaaaaaaaacatacaaagatgggtttctgaataaaatttgtagtgataacagtaaaaaaaaaaaaaaaaaaa

SEQ ID NO: 4 - protein, 240 aa, homo sapiens
  MISC_FEATURE (34)..(34): Xaa can be Ser, Phe or Tyr
  MISC_FEATURE (157)..(157): Xaa can be Gln, Pro or Arg
MAEYLASIFGTEKDKVNCSFYFKIGACRHGDRCXRLHNKPTFSQTIALLNIYRNPQNSSQSADGLRCAVSDVEMQEHYDEFFEEVFTEMEEKYGEVEEMNVCDNLGDHLVGNVYVKFRREEDAEKAVIDLNNRWFNGQPIHAELSPVTDFREACCRXYEMGECTRGGFCNFMHLKPISRELRRELYGRRRKKHRSRSRSRERRSRSRDRGRGGGGGGGGGGGGRERDRRRSRDRERSGRF

SEQ ID NO: 5 - protein, 167 aa, homo sapiens
MQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 6 - protein, 240 aa, homo sapiens
  MISC_FEATURE (34)..(34): Xaa can be Ser, Phe or Tyr
  MISC_FEATURE (157)..(157): Xaa can be Gln, Pro or Arg
MAEYLASIFGTEKDKVNCSFYFKIGACRHGDRCXRLHNKPTFSQTILIQNIYRNPQNSAQTADGSHCAVSDVEMQEHYDEFFEEVFTEMEEKYGEVEEMNVCDNLGDHLVGNVYVKFRREEDAEKAVIDLNNRWFNGQPIHAELSPVTDFREACCRXYEMGECTRGGFCNFMHLKPISRELRRELYGRRRKKHRSRSRSRERRSRSRDRGRGGGGGGGGGGGGRERDRRRSRDRERSGRF

SEQ ID NO: 7 - DNA, 665 bp, homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaa

SEQ ID NO: 8 - DNA, 2141 bp, homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggtaattctttcccacttttctataagtattcagaaatttatttgtattaagttgtctttagccctttgattttttttggcatgcttctgaatttgcttttctttttaaaaatcccccttactatagatttgtatagatgtggttgaagtcacagatgccatattcttcatctttataatgcaagttttctttccaggtagaaggacaagaaaaaaatgagccaatgccatagaaacgcaaagggttaaaggttttacttttacttttgttttgggtacgctttctatacttggatcaaatgtatttttctgctcaaaatactttcaaattattgaaagttttcattccaaatcctagatttttaggattgttacccttgagaaactgtttctcacactagagctccccccatagtgctctaaatatagcttagtaaaggcacctactttgctctaagcatacttaaagttaaaacaaaagtatgtatacgaagagtttgatatttttaatagttggagtctgttttgctaattttagtgtgcaagtcagcacaaggggcatagtgagcctgatggaagctttcctcaggtgagtgagtttcacaatcaaaattaggtgcctgtttcttaaaattagattctgctgcaggtttacatactagacttttcagcctgcatgaattagtagggatattctttttttacttgcataattgtaattgatttaaacatgtatgccagaattcacaggcccatactaactgtccttaatttctttcctacagcagtactgctatatgccagtcctgtctgcattcttaagggtgcagttcaacacatcctctctagattatggtgaaaaagtattccaaaggaagtcttatcagagctagtgtcagaaagaatgacactaccaattgggcatgatcttcagcataggaaatgtcttagaattttattattttttcctaaattgtgatgctatactacttgtatagctagttaccacatttctaaagcatagtgtgcctctgtgaatcttgttatatactgtttgggcttctgtaagatattttgaagtttgaaaagcaaatactgaaactttaaccaagagactgacacatctctttgtacaccacgttgcttctgtgtgcatcataataactggtagaagtaatatatttaaagtaatatgtttccagttttaatttatgctttctaagaaataaaaatatgttttctgggtcaacaaaacttaaagcatgaagccctttttagtaacgtgacatcagaataagattataggtatatttttaaaatcagattttctaaactacaattgttttagaacactgtatgaagaacttatctagtcatagttatttgtagtcaggattttaacagcttgcaacaggtaatgttttgaatataaatatctttccaaagtgttactaatggtaaagagataattttctaggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaa

SEQ ID NO: 9 - DNA, 4338 bp, homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaa

SEQ ID NO: 10 - protein, 154 aa, homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGNSFPLFYKYSEIYLY

SEQ ID NO: 11 - protein, 144 aa, homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGFYSAA

SEQ ID NO: 12 - protein, 1304 aa, homo sapiens
  MISC_FEATURE (622)..(622): Xaa can be Glu or Asp
  MISC_FEATURE (625)..(625): Xaa can be Arg or Leu
  MISC_FEATURE (626)..(626): Xaa can be Asn or His
  MISC_FEATURE (659)..(659): Xaa can be Arg or Gln
  MISC_FEATURE (662)..(662): Xaa can be His , Gln or Asp
  MISC_FEATURE (666)..(666): Xaa can be Lys, Asn, Thr, Glu, Arg or Gln
  MISC_FEATURE (700)..(700): Xaa can be Lys or Glu
  MISC_FEATURE (740)..(740): Xaa can be Gly or Glu
  MISC_FEATURE (741)..(741): Xaa can be Lys or Asn
  MISC_FEATURE (742)..(742): Xaa can be Gly or Asp
  MISC_FEATURE (903)..(903): Xaa can be Gln or Arg
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDXYVXXTTARAFAVVASALGIPSLLPFLKAVCKSKKSWXARXTGIXIVQQIAILMGCAILPHLRSLVEIIEHGLVDEQQXVRTISALAIAALAEAATPYGIESFDSVLKPLWKGIRQHRXXXLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEXTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 13 - DNA, 2008 bp, homo sapiens
agaaggtttcatttccgggtggcgcgggcgccattttgtgaggagcgatataaacgggcgcagaggccggctgcccgcccagttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagctatgagctacggccgcccccctcccgatgtggagggtatgacctccctcaaggtggacaacctgacctaccgcacctcgcccgacacgctgaggcgcgtcttcgagaagtacgggcgcgtcggcgacgtgtacatcccgcgggaccgctacaccaaggagtcccgcggcttcgccttcgttcgctttcacgacaagcgcgacgctgaggacgctatggatgccatggacggggccgtgctggacggccgcgagctgcgggtgcaaatggcgcgctacggccgccccccggactcacaccacagccgccggggaccgccaccccgcaggtacgggggcggtggctacggacgccggagccgcagccctaggcggcgtcgccgcagccgatcccggagtcggagccgttccaggtctcgcagccgatctcgctacagccgctcgaagtctcggtcccgcactcgttctcgatctcggtcgacctccaagtccagatccgcacgaaggtccaagtccaagtcctcgtcggtctccagatctcgttcgcggtccaggtcccggtctcggtccaggagtcctcccccagtgtccaagagggaatccaaatccaggtcgcgatcgaagagtccccccaagtctcctgaagaggaaggagcggtgtcctcttaagaaaatgatgtatcggcaagcagtgtaaacggaggacttggggaaaaaggaccacatagtccatcgaagaagagtccttggaacaagcaactggctattgaaaaggttattttgtaacatttgtctaactttttacttgtttaagctttgcctcagttggcaaacttcattttatgtgccattttgttgctgttattcaaatttcttgtaatttagtgaggtgaacgacttcagatttcattattggatttggatatttgaggtaaaatttcattttgttatatagtgctgactttttttgtttgaaattaaacagattggtaacctaatttgtggcctcctgacttttaaggaaaacgtgtgcagccattacacacagcctaaagctgtcaagagattgactcggcattgccttcattccttaaaattaaaaacctacaaaagttggtgtaaatttgtatatgttatttaccttcagatctaaatggtaatctgaacccaaatttgtataaagacttttcaggtgaaaagacttgattttttgaaaggattgtttatcaaacacaattctaatctcttctcttatgtatttttgtgcactaggcgcagttgtgtagcagttgagtaatgctggttagctgttaaggtggcgtgttgcagtgcagagtgcttggctgtttcctgttttctcccgattgctcctgtgtaaagatgccttgtcgtgcagaaacaaatggctgtccagtttattaaaatgcctgacaactgcacttccagtcacccgggccttgcatataaataacggagcatacagtgagcacatctagctgatgataaatacacctttttttccctcttccccctaaaaatggtaaatctgatcatatctacatgtatgaacttaacatggaaaatgttaaggaagcaaatggttgtaactttgtaagtacttataacatggtgtatctttttgcttatgaatattctgtattataaccattgtttctgtagtttaattaaaacattttcttggtgttagcttttctcagaaaaaaaaaaaaaaaaaaaaaaaaaaaaaaaaaaaaaaaaaa

SEQ ID NO: 14 - DNA, 3003 bp, homo sapiens
agaaggtttcatttccgggtggcgcgggcgccattttgtgaggagcgatataaacgggcgcagaggccggctgcccgcccagttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagctatgagctacggccgcccccctcccgatgtggagggtatgacctccctcaaggtggacaacctgacctaccgcacctcgcccgacacgctgaggcgcgtcttcgagaagtacgggcgcgtcggcgacgtgtacatcccgcgggaccgctacaccaaggagtcccgcggcttcgccttcgttcgctttcacgacaagcgcgacgctgaggacgctatggatgccatggacggggccgtgctggacggccgcgagctgcgggtgcaaatggcgcgctacggccgccccccggactcacaccacagccgccggggaccgccaccccgcaggtacgggggcggtggctacggacgccggagccgcagccctaggcggcgtcgccgcagccgatcccggagtcggagccgttccaggtctcgcagccgatctcgctacagccgctcgaagtctcggtcccgcactcgttctcgatctcggtcgacctccaagtccagatccgcacgaaggtccaagtccaagtcctcgtcggtctccagatctcgttcgcggtccaggtcccggtctcggtccaggagtcctcccccagtgtccaagagggaatccaaatccaggtcgcgatcgaagagtccccccaagtctcctgaagaggaaggagcggtgtcctcttaagaaaatggtaatgtctgggaatccgagacacataaccctaattcataaatgggatttggggtaggtctttttgagtcgtgttaatgtaagaatgactcctatcattaggagtgctgctcggaggttactcacctttgggagtaatactgaagagaggggtctgcagaaaggatgtgtatgaagcttagataataatggctgtttcgtaaactgtttgagacctattaatgaaaatgactatttcttgctgtttttatccaacgtctgcattttccccctttaaagctgcggtctcctgtttgataaaagaatattggccagtattgcagattttaactgatttggctgatcctccagggaccagtttctgtgggcgtgtattggagcaggtttgtctttaaatgttaaagatgcactatcctcttagagaaacaatcagttcaactattgttgtactgactgggacttcatattctaatggatgtggcaaaagaattgcaataagaagcagtgaacatttggaaccccaaaagaaagttacaggtattgcactgggtggggaaaggatagtgtgtctttaactcttaaattgtttggtcctattttttaaaaaggaaagggccctaagtagctcagatattaaagtagtattctcaattaccaaatgtttcatttgaaacaatttatcttaatgaaatatagaccaattctctgatctcgagttgtttttgtttggatacagcccttttttttttctttttttttcttccccttacctttcttcaccttggttatttggccaggaatacgtaaattcaaacttgtacatgctgatggtagcctttgtgaaattttcctaattgggccttttaaaaacatggctgggtggaacatttctgtaccctactggtttgaccagagccttagtaagtacgtgcctgaaactgaaaccatgtgcactttaatggaaggtaagctgaacttctttcttttcaaacctagatgtatcggcaagcagtgtaaacggaggacttggggaaaaaggaccacatagtccatcgaagaagagtccttggaacaagcaactggctattgaaaaggttattttgtaacatttgtctaactttttacttgtttaagctttgcctcagttggcaaacttcattttatgtgccattttgttgctgttattcaaatttcttgtaatttagtgaggtgaacgacttcagatttcattattggatttggatatttgaggtaaaatttcattttgttatatagtgctgactttttttgtttgaaattaaacagattggtaacctaatttgtggcctcctgacttttaaggaaaacgtgtgcagccattacacacagcctaaagctgtcaagagattgactcggcattgccttcattccttaaaattaaaaacctacaaaagttggtgtaaatttgtatatgttatttaccttcagatctaaatggtaatctgaacccaaatttgtataaagacttttcaggtgaaaagacttgattttttgaaaggattgtttatcaaacacaattctaatctcttctcttatgtatttttgtgcactaggcgcagttgtgtagcagttgagtaatgctggttagctgttaaggtggcgtgttgcagtgcagagtgcttggctgtttcctgttttctcccgattgctcctgtgtaaagatgccttgtcgtgcagaaacaaatggctgtccagtttattaaaatgcctgacaactgcacttccagtcacccgggccttgcatataaataacggagcatacagtgagcacatctagctgatgataaatacacctttttttccctcttccccctaaaaatggtaaatctgatcatatctacatgtatgaacttaacatggaaaatgttaaggaagcaaatggttgtaactttgtaagtacttataacatggtgtatctttttgcttatgaatattctgtattataaccattgtttctgtagtttaattaaaacattttcttggtgttagcttttctcagaaaaaaaaaaaaaaaaaaaaaaaaaaaaaaaaaaaaaaaaaa

SEQ ID NO: 15 - DNA, 2606 bp, homo sapiens
cagttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagctatgagctacggccgcccccctcccgatgtggagggtatgacctccctcaaggtggacaacctgacctaccgcacctcgcccgacacgctgaggcgcgtcttcgagaagtacgggcgcgtcggcgacgtgtacatcccgcgggaccgctacaccaaggagtcccgcggcttcgccttcgttcgctttcacgacaagcgcgacgctgaggacgctatggatgccatggacggggccgtgctggacggccgcgagctgcgggtgcaaatggcgcgctacggccgccccccggactcacaccacagccgccggggaccgccaccccgcaggtacgggggcggtggctacggacgccggagccgcagccctaggcggcgtcgccgcagccgatcccggagtcggagtcgttccaggtctcgcagccgatctcgctacagccgctcgaagtctcggtcccgcactcgttctcgatctcggtcgacctccaagtccagatccgcacgaaggtccaagtccaagtcctcgtcggtctccagatctcgttcgcggtccaggtcccggtctcggtccaggagtcctcccccagtgtccaagagggaatccaaatccaggtcgcgatcgaagagtccccccaagtctcctgaagaggaaggagcggtgtcctcttaagaaaatgctgcggtctcctgtttgataaaagaatattggccagtattgcagattttaactgatttggctgatcctccagggaccagtttctgtgggcgtgtattggagcaggtttgtctttaaatgttaaagatgcactatcctcttagagaaacaatcagttcaactattgttgtactgactgggacttcatattctaatggatgtggcaaaagaattgcaataagaagcagtgaacatttggaaccccaaaagaaagttacaggtattgcactgggtggggaaaggatagtgtgtctttaactcttaaattgtttggtcctattttttaaaaaggaaagggccctaagtagctcagatattaaagtagtattctcaattaccaaatgtttcatttgaaacaatttatcttaatgaaatatagaccaattctctgatctcgagttgtttttgtttggatacagcccttttttttttctttttttttcttccccttacctttcttcaccttggttatttggccaggaatacgtaaattcaaacttgtacatgctgatggtagcctttgtgaaattttcctaattgggccttttaaaaacatggctgggtggaacatttctgtaccctactggtttgaccagagccttagtaagtacgtgcctgaaactgaaaccatgtgcactttaatggaaggtaagctgaacttctttcttttcaaacctagatgtatcggcaagcagtgtaaacggaggacttggggaaaaaggaccacatagtccatcgaagaagagtccttggaacaagcaactggctattgaaaaggttattttgtaacatttgtctaactttttacttgtttaagctttgcctcagttggcaaacttcattttatgtgccattttgttgctgttattcaaatttcttgtaatttagtgaggtgaacgacttcagatttcattattggatttggatatttgaggtaaaatttcattttgttatatagtgctgactttttttgtttgaaattaaacagattggtaacctaatttgtggcctcctgacttttaaggaaaacgtgtgcagccattacacacagcctaaagctgtcaagagattgactcagcattgccttcattccttaaaattaaaaacctacaaaagttggtgtaaatttgtatatgttatttaccttcagatctaaatggtaatctgaacccaaatttgtataaagacttttcaggtgaaaagacttgattttttgaaaggattgtttatcaaacacaattctaatctcttctcttatgtatttttgtgcactaggcgcagttgtgtagcagttgagtaatgctggttagctgttaaggtggcgtgttgcagtgcagagtgcttggctgtttcctgttttctcccgattgctcctgtgtaaagatgccttgtcgtgcagaaacaaatggctgtccagtttattaaaatgcctgacaactgcacttccagtcacccgggccttgcatataaataacggagcatacagtgagcacatctagctgatgataaatacacctttttttccctcttccccctaaaaatggtaaatctgatcatatctacatgtatgaacttaacatggaaaatgttaaggaagcaaatggttgtaactttgtaagtacttataacatggtgtatctttttgcttatgaatattctgtattataaccattgtttctgtagtttaattaaaacattttcttggtgttagcttttctcagaa

SEQ ID NO: 16 - protein, 221 aa, homo sapiens
  MISC_FEATURE (95)..(95): Xaa can be Pro, His , Leu or Arg
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRXPDSHHSRRGPPPRRYGGGGYGRRSRSPRRRRRSRSRSRSRSRSRSRSRYSRSKSRSRTRSRSRSTSKSRSARRSKSKSSSVSRSRSRSRSRSRSRSPPPVSKRESKSRSRSKSPPKSPEEEGAVSS

SEQ ID NO: 17 - protein, 221 aa, homo sapiens; identical to SEQ ID NO: 16
  MISC_FEATURE (95)..(95): Xaa can be Pro, His , Leu or Arg

SEQ ID NO: 18 - protein, 221 aa, homo sapiens; identical to SEQ ID NO: 16
  MISC_FEATURE (95)..(95): Xaa can be Pro, His , Leu or Arg

SEQ ID NO: 19 - DNA, 1512 bp, homo sapiens
cgccctttcctgccagacctggaggggcggggcggtgccggcaagatggctgcgcccgagaagatgacgtttcccgagaaaccaagccacaaaaagtacagggccgccctgaagaaggagaaacgaaagaaacgtcggcaggaacttgctcgactgagagactcaggactctcacagaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcgggtaatagagacagaactgttcagagtcccaaatccaaataaactagttttgttcttaaa

SEQ ID NO: 20 - DNA, 3987 bp, homo sapiens
gaggggcggggcggtgccggcaagatggctgcgcccgagaagatgacgtttcccgagaaaccaagccacaaaaagtacagggccgccctgaagaaggagaaacgaaagaaacgtcggcaggaacttgctcgactgagagactcagaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcggagacagggtcttgctatgttgcccagactggagggcagtggctgttcacatgatcattgtgcactgtagcctcaaactcctgggctcaagcgatcttcttggctcagcctcctgaatatctgaaactacaggtgagccctgcttgtttgccttttattaataatctattggatttttctatatgtaatagctgaacagcttttttgctttaaagttcattttctcagttttaaagtactgatgggcaaagtccattttttttcctcctactcttcagctgggggctgtcgtgtacttttttaaatagtgtgttggaaagttctttatataggagtaaagtttatttttgtcatttccaggaatattagggtcattggtactaaatatgcctagtgttaaagcaatcctggtataaaaatctcaacagaatggtcagagaatagaacctaatgcagaaaaagcttcaggcaaaatattatacctagatttctgatcataaataagatgctctccagctttatcaattttttgtcgctttctttccattgacatatttgggatctagaattttggagtatgtgacatttttcaatttgagatttgggaataaggacttaaaacatacgtaaaatactgaaacgttaacgaaatttaagtaggaagaaagtttcctcaaaattggaatttctgcatgatatctgttaaggatgagagttctttcatgtgtagtcaataaaagaaatcatgcatgtggcccaggataatttcactttaaaaagttggatttgttccagactctaaatgcctgcttcaccctgaaaatgattgacctagaactatctactgatttcagtaacgccgagtaattatcattaaattcagtattttttaatacttttctaaatacagtcaagatatatgcaattaaatgtggtctgcaaagctgaaatcttaataaatcatggaggctaagaaagaaaaccaccatcttgagctcaggacaactgagatattgatgactttcccctcaaatgttaataggcagggtagaagggcaggaagtttttaaaccactgactggttcattatcctgataaatgtccatcttgaacttaaatttattagttttttttaatatatattgcctgattactatttatgtttcaaaaattccaactatacattgctagcagaaacccacaggcctgttttcagtgaatagctagctgcatgttggaggctttttaaacctataaacgtctttttttccagtagactagcaacttaatttgatggtagttcctgcctccttcttaaatagcccagttttagaatgtaacatttcctatctgtagtaataggaggtcccttaactatcatgattaccatagatttgatttgttgcataggttaggtagtacagtctgcaagtattagtccctttctgttgcatgtgctagcaccttcagttcacaagggggtaagaggaatccagttgatttgaaataccttttgagtaacagtctaagttgttcattcagaaagagtagaaaagtcaactgcatcaaggttttaaaagcagcatttattgattgaaaataaatgtgtagataggctctcagtatggaatccatgttattttttaatgaagtacatgaagactccttagatcttccaccatgtatcttgtgtgtgcttataacaaccaccatattcaaatggaggggaattttcaacattttactgaaaaaaaaatgagaaattcttccttcagcagctctgcatagtttgacaaacttttggaaagagataaaaacacactccagtgtttgtacgttgaacgtttattacaactaattggcgatgtgataagacagtgctcacgtggcctgaatgttggtcacaatcacaacaaagcttaatccagcccagcatatataagtgaaaatataaaccatgaagacatgtttagatatgtataagtacttagaaaagtgacgctgaacaattacatagctttaaaaaatatagagccatctaatgccattttacctcattcaactcttttttgtaagaaatgtgtctttttaaatttttataagacttctgttaactaggcagtgccatggaaagaaaggaagctgctgtaaagtgtgcagcacttctaagtggagggaagcaaaataaatttagctttaacaagtaccccagagggaacattcgtgctttaaacaatgactttacacataatactgtgaagagagcaacacgttttttgtaagatactgttatgaagaattcctggcaaattaccattctcactttattttggggcatcaaaaacaactaagtatttgattatgaccacaaaaaattcatctcaggagtcaaatgtgccttggccttaaaaattatgtgcttactattgtgaaacaatcctgtttctcatcccagattcagaatgcctaagaaataatttttagtgtaagaagtatcactccaatttagtcattaaaaataatctgtagtcctttaaaaaaaaaaaaaaaaggtatgtaatttccaa

SEQ ID NO: 21 - DNA, 3951 bp, homo sapiens
cctcagcctccagagtagttgggattacagacacgcaccaccgcgcccggccatcatgacttttctctgcttcttgagagcacttccagcatcgctagtcgcactttgtaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcggagacagggtcttgctatgttgcccagactggagggcagtggctgttcacatgatcattgtgcactgtagcctcaaactcctgggctcaagcgatcttcttggctcagcctcctgaatatctgaaactacaggtgagccctgcttgtttgccttttattaataatctattggatttttctatatgtaatagctgaacagcttttttgctttaaagttcattttctcagttttaaagtactgatgggcaaagtccattttttttcctcctactcttcagctgggggctgtcgtgtacttttttaaatagtgtgttggaaagttctttatataggagtaaagtttatttttgtcatttccaggaatattagggtcattggtactaaatatgcctagtgttaaagcaatcctggtataaaaatctcaacagaatggtcagagaatagaacctaatgcagaaaaagcttcaggcaaaatattatacctagatttctgatcataaataagatgctctccagctttatcaattttttgtcgctttctttccattgacatatttgggatctagaattttggagtatgtgacatttttcaatttgagatttgggaataaggacttaaaacatacgtaaaatactgaaacgttaacgaaatttaagtaggaagaaagtttcctcaaaattggaatttctgcatgatatctgttaaggatgagagttctttcatgtgtagtcaataaaagaaatcatgcatgtggcccaggataatttcactttaaaaagttggatttgttccagactctaaatgcctgcttcaccctgaaaatgattgacctagaactatctactgatttcagtaacgccgagtaattatcattaaattcagtattttttaatacttttctaaatacagtcaagatatatgcaattaaatgtggtctgcaaagctgaaatcttaataaatcatggaggctaagaaagaaaaccaccatcttgagctcaggacaactgagatattgatgactttcccctcaaatgttaataggcagggtagaagggcaggaagtttttaaaccactgactggttcattatcctgataaatgtccatcttgaacttaaatttattagttttttttaatatatattgcctgattactatttatgtttcaaaaattccaactatacattgctagcagaaacccacaggcctgttttcagtgaatagctagctgcatgttggaggctttttaaacctataaacgtctttttttccagtagactagcaacttaatttgatggtagttcctgcctccttcttaaatagcccagttttagaatgtaacatttcctatctgtagtaataggaggtcccttaactatcatgattaccatagatttgatttgttgcataggttaggtagtacagtctgcaagtattagtccctttctgttgcatgtgctagcaccttcagttcacaagggggtaagaggaatccagttgatttgaaataccttttgagtaacagtctaagttgttcattcagaaagagtagaaaagtcaactgcatcaaggttttaaaagcagcatttattgattgaaaataaatgtgtagataggctctcagtatggaatccatgttattttttaatgaagtacatgaagactccttagatcttccaccatgtatcttgtgtgtgcttataacaaccaccatattcaaatggaggggaattttcaacattttactgaaaaaaaaatgagaaattcttccttcagcagctctgcatagtttgacaaacttttggaaagagataaaaacacactccagtgtttgtacgttgaacgtttattacaactaattggcgatgtgataagacagtgctcacgtggcctgaatgttggtcacaatcacaacaaagcttaatccagcccagcatatataagtgaaaatataaaccatgaagacatgtttagatatgtataagtacttagaaaagtgacgctgaacaattacatagctttaaaaaatatagagccatctaatgccattttacctcattcaactcttttttgtaagaaatgtgtctttttaaatttttataagacttctgttaactaggcagtgccatggaaagaaaggaagctgctgtaaagtgtgcagcacttctaagtggagggaagcaaaataaatttagctttaacaagtaccccagagggaacattcgtgctttaaacaatgactttacacataatactgtgaagagagcaacacgttttttgtaagatactgttatgaagaattcctggcaaattaccattctcactttattttggggcatcaaaaacaactaagtatttgattatgaccacaaaaaattcatctcaggagtcaaatgtgccttggccttaaaaattatgtgcttactattgtgaaacaatcctgtttctcatcccagattcagaatgcctaagaaataatttttagtgtaagaagtatcactccaatttagtcattaaaaataatctgtagtcctttaaaaaaaaaaaaaaaaggtatgtaatttccaa

SEQ ID NO: 22 - DNA, 3986 bp, homo sapiens
ggtgccggcaagatggctgcgcccgagaagatgacgtttcccgagaaaccaagccacaaaaagtacagggccgccctgaagaaggagaaacgaaagaaacgtcggcaggaacttgctcgactgagagactcaggactctcacagaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcggagacagggtcttgctatgttgcccagactggagggcagtggctgttcacatgatcattgtgcactgtagcctcaaactcctgggctcaagcgatcttcttggctcagcctcctgaatatctgaaactacaggtgagccctgcttgtttgccttttattaataatctattggatttttctatatgtaatagctgaacagcttttttgctttaaagttcattttctcagttttaaagtactgatgggcaaagtccattttttttcctcctactcttcagctgggggctgtcgtgtacttttttaaatagtgtgttggaaagttctttatataggagtaaagtttatttttgtcatttccaggaatattagggtcattggtactaaatatgcctagtgttaaagcaatcctggtataaaaatctcaacagaatggtcagagaatagaacctaatgcagaaaaagcttcaggcaaaatattatacctagatttctgatcataaataagatgctctccagctttatcaattttttgtcgctttctttccattgacatatttgggatctagaattttggagtatgtgacatttttcaatttgagatttgggaataaggacttaaaacatacgtaaaatactgaaacgttaacgaaatttaagtaggaagaaagtttcctcaaaattggaatttctgcatgatatctgttaaggatgagagttctttcatgtgtagtcaataaaagaaatcatgcatgtggcccaggataatttcactttaaaaagttggatttgttccagactctaaatgcctgcttcaccctgaaaatgattgacctagaactatctactgatttcagtaacgccgagtaattatcattaaattcagtattttttaatacttttctaaatacagtcaagatatatgcaattaaatgtggtctgcaaagctgaaatcttaataaatcatggaggctaagaaagaaaaccaccatcttgagctcaggacaactgagatattgatgactttcccctcaaatgttaataggcagggtagaagggcaggaagtttttaaaccactgactggttcattatcctgataaatgtccatcttgaacttaaatttattagttttttttaatatatattgcctgattactatttatgtttcaaaaattccaactatacattgctagcagaaacccacaggcctgttttcagtgaatagctagctgcatgttggaggctttttaaacctataaacgtctttttttccagtagactagcaacttaatttgatggtagttcctgcctccttcttaaatagcccagttttagaatgtaacatttcctatctgtagtaataggaggtcccttaactatcatgattaccatagatttgatttgttgcataggttaggtagtacagtctgcaagtattagtccctttctgttgcatgtgctagcaccttcagttcacaagggggtaagaggaatccagttgatttgaaataccttttgagtaacagtctaagttgttcattcagaaagagtagaaaagtcaactgcatcaaggttttaaaagcagcatttattgattgaaaataaatgtgtagataggctctcagtatggaatccatgttattttttaatgaagtacatgaagactccttagatcttccaccatgtatcttgtgtgtgcttataacaaccaccatattcaaatggaggggaattttcaacattttactgaaaaaaaaatgagaaattcttccttcagcagctctgcatagtttgacaaacttttggaaagagataaaaacacactccagtgtttgtacgttgaacgtttattacaactaattggcgatgtgataagacagtgctcacgtggcctgaatgttggtcacaatcacaacaaagcttaatccagcccagcatatataagtgaaaatataaaccatgaagacatgtttagatatgtataagtacttagaaaagtgacgctgaacaattacatagctttaaaaaatatagagccatctaatgccattttacctcattcaactcttttttgtaagaaatgtgtctttttaaatttttataagacttctgttaactaggcagtgccatggaaagaaaggaagctgctgtaaagtgtgcagcacttctaagtggagggaagcaaaataaatttagctttaacaagtaccccagagggaacattcgtgctttaaacaatgactttacacataatactgtgaagagagcaacacgttttttgtaagatactgttatgaagaattcctggcaaattaccattctcactttattttggggcatcaaaaacaactaagtatttgattatgaccacaaaaaattcatctcaggagtcaaatgtgccttggccttaaaaattatgtgcttactattgtgaaacaatcctgtttctcatcccagattcagaatgcctaagaaataatttttagtgtaagaagtatcactccaatttagtcattaaaaataatctgtagtcctttaaaaaaaaaaaaaaaaggtatgtaatttccaa

SEQ ID NO: 23 - DNA, 3941 bp, homo sapiens
ggattacagacacgcaccaccgcgcccggccatcatgacttttctctgcttcttgagagcacttccagcatcgctagtcgcactttgtgactctcacagaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcggagacagggtcttgctatgttgcccagactggagggcagtggctgttcacatgatcattgtgcactgtagcctcaaactcctgggctcaagcgatcttcttggctcagcctcctgaatatctgaaactacaggtgagccctgcttgtttgccttttattaataatctattggatttttctatatgtaatagctgaacagcttttttgctttaaagttcattttctcagttttaaagtactgatgggcaaagtccattttttttcctcctactcttcagctgggggctgtcgtgtacttttttaaatagtgtgttggaaagttctttatataggagtaaagtttatttttgtcatttccaggaatattagggtcattggtactaaatatgcctagtgttaaagcaatcctggtataaaaatctcaacagaatggtcagagaatagaacctaatgcagaaaaagcttcaggcaaaatattatacctagatttctgatcataaataagatgctctccagctttatcaattttttgtcgctttctttccattgacatatttgggatctagaattttggagtatgtgacatttttcaatttgagatttgggaataaggacttaaaacatacgtaaaatactgaaacgttaacgaaatttaagtaggaagaaagtttcctcaaaattggaatttctgcatgatatctgttaaggatgagagttctttcatgtgtagtcaataaaagaaatcatgcatgtggcccaggataatttcactttaaaaagttggatttgttccagactctaaatgcctgcttcaccctgaaaatgattgacctagaactatctactgatttcagtaacgccgagtaattatcattaaattcagtattttttaatacttttctaaatacagtcaagatatatgcaattaaatgtggtctgcaaagctgaaatcttaataaatcatggaggctaagaaagaaaaccaccatcttgagctcaggacaactgagatattgatgactttcccctcaaatgttaataggcagggtagaagggcaggaagtttttaaaccactgactggttcattatcctgataaatgtccatcttgaacttaaatttattagttttttttaatatatattgcctgattactatttatgtttcaaaaattccaactatacattgctagcagaaacccacaggcctgttttcagtgaatagctagctgcatgttggaggctttttaaacctataaacgtctttttttccagtagactagcaacttaatttgatggtagttcctgcctccttcttaaatagcccagttttagaatgtaacatttcctatctgtagtaataggaggtcccttaactatcatgattaccatagatttgatttgttgcataggttaggtagtacagtctgcaagtattagtccctttctgttgcatgtgctagcaccttcagttcacaagggggtaagaggaatccagttgatttgaaataccttttgagtaacagtctaagttgttcattcagaaagagtagaaaagtcaactgcatcaaggttttaaaagcagcatttattgattgaaaataaatgtgtagataggctctcagtatggaatccatgttattttttaatgaagtacatgaagactccttagatcttccaccatgtatcttgtgtgtgcttataacaaccaccatattcaaatggaggggaattttcaacattttactgaaaaaaaaatgagaaattcttccttcagcagctctgcatagtttgacaaacttttggaaagagataaaaacacactccagtgtttgtacgttgaacgtttattacaactaattggcgatgtgataagacagtgctcacgtggcctgaatgttggtcacaatcacaacaaagcttaatccagcccagcatatataagtgaaaatataaaccatgaagacatgtttagatatgtataagtacttagaaaagtgacgctgaacaattacatagctttaaaaaatatagagccatctaatgccattttacctcattcaactcttttttgtaagaaatgtgtctttttaaatttttataagacttctgttaactaggcagtgccatggaaagaaaggaagctgctgtaaagtgtgcagcacttctaagtggagggaagcaaaataaatttagctttaacaagtaccccagagggaacattcgtgctttaaacaatgactttacacataatactgtgaagagagcaacacgttttttgtaagatactgttatgaagaattcctggcaaattaccattctcactttattttggggcatcaaaaacaactaagtatttgattatgaccacaaaaaattcatctcaggagtcaaatgtgccttggccttaaaaattatgtgcttactattgtgaaacaatcctgtttctcatcccagattcagaatgcctaagaaataatttttagtgtaagaagtatcactccaatttagtcattaaaaataatctgtagtcctttaaaaaaaaaaaaaaaaggtatgtaatttccaa

SEQ ID NO: 24 - protein, 348 aa, homo sapiens
MLDQAENELENGTTWQNPEPPVDFRVMEKDRANCPFYSKTGACRFGDRCSRKHNFPTSSPTLLIKSMFTTFGMEQCRRDDYDPDASLEYSEEETYQQFLDFYEDVLPEFKNVGKVIQFKVSCNLEPHLRGNVYVQYQSEEECQAALSLFNGRWYAGRQLQCEFCPVTRWKMAICGLFEIQQCPRGKHCNFLHVFRNPNNEFWEANRDIYLSPDRTGSSFGKNSERRERMGHHDDYYSRLRGRRNPSPDHSYKRNGESERKSSRHRGKKSHKRTSKSRERHNSRSRGRNRDRSRDRSRGRGSRSRSRSRSRRSRRSRSQSSSRSRSRGRRRSETGSCYVAQTGGQWLFT

SEQ ID NO: 25 - protein, 348 aa, homo sapiens; identical to SEQ ID NO: 24

SEQ ID NO: 26 - protein, 348 aa, homo sapiens; identical to SEQ ID NO: 24

SEQ ID NO: 27 - protein, 482 aa, homo sapiens
MAAPEKMTFPEKPSHKKYRAALKKEKRKKRRQELARLRDSGLSQKEEEEDTFIEEQQLEEEKLLERERQRLHEEWLLREQKAQEEFRIKKEKEEAAKKRQEEQERKLKEQWEEQQRKEREEEEQKRQEKKEKEEALQKMLDQAENELENGTTWQNPEPPVDFRVMEKDRANCPFYSKTGACRFGDRCSRKHNFPTSSPTLLIKSMFTTFGMEQCRRDDYDPDASLEYSEEETYQQFLDFYEDVLPEFKNVGKVIQFKVSCNLEPHLRGNVYVQYQSEEECQAALSLFNGRWYAGRQLQCEFCPVTRWKMAICGLFEIQQCPRGKHCNFLHVFRNPNNEFWEANRDIYLSPDRTGSSFGKNSERRERMGHHDDYYSRLRGRRNPSPDHSYKRNGESERKSSRHRGKKSHKRTSKSRERHNSRSRGRNRDRSRDRSRGRGSRSRSRSRSRRSRRSRSQSSSRSRSRGRRRSGNRDRTVQSPKSK

SEQ ID NO: 28 - protein, 348 aa, homo sapiens; identical to SEQ ID NO: 24

SEQ ID NO: 29 - DNA, 2314 bp, homo sapiens
aggggccggttgctccggaagtggagggagggggtgaaaatggcgcccagctcgaaatcggagcggaacagcggggctgggagcggcggcggcggccccgggggagccggagggaagcgggcagcagggcggcggcgggagcacgtcctcaagcagctggagcgggtcaagatcagcggacagctctcccctcgcctcttccggaagctgcctccccgggtgtgcgtgtccctcaagaacattgtggatgaggacttcctctatgcaggacatatcttcctgggcttttccaaatgcggccgctacgtcctctcctacaccagcagcagtggggatgacgacttctccttctacatctaccatctgtactggtgggagttcaacgttcacagcaagctcaagctggtccggcaggttcggctattccaggacgaggagatctacagcgacctgtacctgaccgtatgcgagtggcccagcgacgcctccaaggtcatcgtcttcggcttcaacacccgctcggccaacgggatgctcatgaacatgatgatgatgagtgacgagaaccaccgtgacatctacgtcagcaccgtggccgtgccaccgccaggccgctgtgctgcttgccaggatgccagccgagcccacccaggagacccgaatgcacagtgcctacggcatggcttcatgctgcacaccaagtaccaggtggtctaccccttccccaccttccagcccgccttccagctcaagaaggaccaggtggtgctgctcaacaccagctactccctggtggcctgcgccgtctccgtccactcggcaggtgacaggagtttctgccaaatcctgtatgaccacagcacctgccccctggcgcctgccagcccccctgagccccagagcccagagctgccccctgccctccccagcttctgccctgaggcggccccagcccgttcttctgggtctcctgagccctcgcccgccattgccaaagccaaggagtttgtggctgacatcttccgccgggccaaagaggccaagggcggggtccctgaggaagcccggcctgccctgtgcccaggaccctctggcagccgctgccgtgcgcactctgagcccctagccctgtgtggagagacggcaccccgggacagcccccctgcctcggaggcacctgcctccgagcctggctatgtcaactacaccaagctgtactatgtgctggagtccggagaggggacggagccggaggatgagttggaggacgacaagatctccctgcccttcgtggtgactgatcttcgtggccgcaacctgcggcccatgcgggagcggactgctgtccagggccagtacctgacagtggagcagctcacactagacttcgaatatgttatcaatgaggtcatccgccacgacgctacctggggccatcagttctgttctttcagcgactatgacatcgtcattctggaggtctgcccagaaaccaaccaggtcctcatcaacattggcctgctgctcctggccttcccgtcccccactgaggagggccagctccgaccaaagacctatcacaccagcctcaaggtggcatgggacctcaacacagggatcttcgagacagtcagtgtaggcgacctgactgaggtcaaagggcagaccagcggcagtgtctggagctcctaccgcaagagctgcgtggacatggtcatgaagtggctggtgccggagagcagcggccgctacgtcaacaggatgaccaatgaggcgctgcacaaagggtgctccctgaaggttctggcggacagcgagcgatatacgtggatcgtgctgtgagggccaggccgccccggacactgactccaactacctccgtggcctgggaccggcccccttcctggggtggcctcttcctggccggctggcccaccgactgatgaccggcactagtgttagcctgcggaacggggctgggcagggcagcctctgttggcctgagggtctggacgctttttatttatgcctatttaagttgggaaggggcagagagagggcgccccctgccccaccagcctgagtgccccgccttcaccccgagctgggcatgggcctggcccctcgtgcatttgcccttttctcggctacagctgtggacgttgccctcggggaggtcgaatggaccccattccccctgccctgcccgcccccagcctccccacccaggccggcaacctggccatccccattccgttcttcttcatgtaataaatgttttaatttctgaacctcaaaaaaaaaaaaaaaaaaa

SEQ ID NO: 30 - protein, 600 aa, homo sapiens
MAPSSKSERNSGAGSGGGGPGGAGGKRAAGRRREHVLKQLERVKISGQLSPRLFRKLPPRVCVSLKNIVDEDFLYAGHIFLGFSKCGRYVLSYTSSSGDDDFSFYIYHLYWWEFNVHSKLKLVRQVRLFQDEEIYSDLYLTVCEWPSDASKVIVFGFNTRSANGMLMNMMMMSDENHRDIYVSTVAVPPPGRCAACQDASRAHPGDPNAQCLRHGFMLHTKYQVVYPFPTFQPAFQLKKDQVVLLNTSYSLVACAVSVHSAGDRSFCQILYDHSTCPLAPASPPEPQSPELPPALPSFCPEAAPARSSGSPEPSPAIAKAKEFVADIFRRAKEAKGGVPEEARPALCPGPSGSRCRAHSEPLALCGETAPRDSPPASEAPASEPGYVNYTKLYYVLESGEGTEPEDELEDDKISLPFVVTDLRGRNLRPMRERTAVQGQYLTVEQLTLDFEYVINEVIRHDATWGHQFCSFSDYDIVILEVCPETNQVLINIGLLLLAFPSPTEEGQLRPKTYHTSLKVAWDLNTGIFETVSVGDLTEVKGQTSGSVWSSYRKSCVDMVMKWLVPESSGRYVNRMTNEALHKGCSLKVLADSERYTWIVL

SEQ ID NO: 31 - DNA, 5042 bp, homo sapiens
atgtgtgctggtgaatgtgagtacagggaagcagcggccgccatttcagggagcttgtcgacgctgtcgcaggggtggatcctgagctgccgaagccgccgtcctgctctcccgcgtgggcttctctaattccattgttttttttagattctctcgggcctagccgtccttggaacccgatattcgggctgggcggttccgcggcctgggcctaggggcttaacagtagcaacagaagcggcggcggcggcagcagcagcagcagcagcagcaatctcttcccgaacacgagcaccacaggcgcccgaaggccggaacaggcgtttagagaaaatggcagacgatattgatattgaagcaatgcttgaggctccttacaagaaggatgagaacaagttgagcagtgccaacggccatgaagaacgtagcaaaaagaggaaaaaaagcaagagcagaagtcgtagtcatgaacgaaagagaagcaaaagtaaggaacggaagcgaagtagagacagagaaaggaaaaagagcaaaagccgtgaaagaaagcgaagtagaagcaaagagaggcgacggagccgctcaagaagtcgagatcgaagatttagaggccgctacagaagtccttactccggaccaaaatttaacagtgccatccgaggaaagattgggttgcctcatagcatcaaattaagcagacgacgttcccgaagcaaaagtccattcagaaaagacaagagccctgtgagagaacctattgataatttaactcctgaggaaagagatgcaaggacagtcttctgtatgcagctggcggcaagaattcgaccaagggatttggaagagtttttctctacagtaggaaaggttcgagatgtgaggatgatttctgacagaaattcaagacgttccaaaggaattgcttatgtggagttcgtcgatgttagctcagtgcctctagcaataggattaactggccaacgagttttaggcgtgccaatcatagtacaggcatcacaggcagaaaaaaacagagctgcagcaatggcaaacaatttacaaaagggaagtgctggacctatgaggctttatgtgggctcattacacttcaacataactgaagatatgcttcgtgggatctttgagccttttggaagaattgaaagtatccagctgatgatggacagtgaaactggtcgatccaagggatatggatttattacattttctgactcagaatgtgccaaaaaggctttggaacaacttaatggatttgaactagcaggaagaccaatgaaagttggtcatgttactgaacgtactgatgcttcgagtgctagttcatttttggacagtgatgaactggaaaggactggaattgatttgggaacaactggtcgtcttcagttaatggcaagacttgcagagggtacaggtttgcagattccgccagcagcacagcaagctctacagatgagtggctctttggcatttggtgctgtggcagatttgcaaacaagactttcccagcagactgaagcttcagctttagctgcagctgcctctgttcagccacttgcaacacaatgtttccaactctctaacatgtttaaccctcaaacagaagaagaagttggatgggataccgagattaaggatgatgtgattgaagaatgtaataaacatggaggagttattcatatttatgttgacaaaaattcagctcagggcaatgtgtatgtgaagtgcccatcaattgctgcagctattgctgctgtcaatgcattgcatggcaggtggtttgctggtaaaatgataacagcagcatatgtacctcttccaacttaccacaacctgtttcctgattctatgacagcaacacagctactggttccaagtagacgatgaaggaagatatagtcccttatgtatatagctttttttctttcttgagaattcatcttgagttatcttttatttagataaaaataaagaggcaaggatctactgtcatttgtatgcaatttcctgttaccttgaaaaaataaaaatgttaacaggaatgcagtgtgctcattctccctaaatagtaaatcccactgtatacaaaactgttctcttgttctgccttttaaaatgttcatgtagaaaattaatgaactataggaatagctctaggagaacaaatgtgctttctgtaaaaaggcagaccagggatgtaatgtttttaatgtttcagaagcctaactttttacacagtggttacatttcacatttcactaatgttgatatttggctgatggttgagcagtttctgaaatacacatttagtgtatggaaatacaagacagctaaagggctgtttggttagcatctcatcttgcattctgatcaattggcaagaaagggagatttcaaaattatatttcttgatggtatcttttcaattaatgtatctgtaaaagtttctttgtaaatactatgtgttctggtgtgtcttaaaattccaaacaaaatgatccctgcatttcctgaagatgtttaaacgtgagagtctggtaggcaaagcagtctgagaaagaaataggaaatgcagaaataggttttgtctggttgcatataatctttgctctttttaagctctgtgagctctgaaatatatttttgggttacttcagtgtgtttgacaagacagcttgatatttctatcaaacaaatgactttcatattgcaacaatctttgtaagaaccactcaaataaaagtctcttaaaaaggccacaggagatcttcatttttcaaatgttttaaagttacagaaatttgagaacagaattagcttcttttagtctcaattcagtacttgcctcttgggaaaatgttccgagtctgcggaaacttgccctcacgttgtcccccatgaattcctttattctattagagtctagccctgtgtattttagaagaatgtatcaaaactgaggggtttaccaaaaagaaaaggaaacagcctggccttaaaacctgggcattcttggtctcctgcattgtctgggcatatttatgaagaaattttcaccatttacaatacatacttaacaaaatggcttaacttcaaactggtttccaaaattgatttatatattaatatattaaataaggtgcatatattactgtattaaggggttttgagctttcatactagttgggaagcaagctgtcaccatttatactcaattctcttcaactgtttatctcttggtaaatcttttgagtaggaaatgctgctcctagcttatcttcaaatactgagttccttacctcatttgttcaacttttttttttgttagcattgaagcacaagttttacaggcttattcagaatttcatatcagtcgttttgtttgacatttatcccaataattgttctagtgatttaatttgtatggaaactcccaaggttcaatttctttttctttttgagatggagtcttgctctttggcccaggctggagtgcagtgatgtgatctcagctccatctcccaggttcaagcaattctcccacctcagtctcctgaagagctgggacaggtgcgcaccaccgtgcccagctaatttttgtatttttagcagagacggtgttagcccggctggtctcacactgacctcaagtgatctgtccgccttggcctcccaaagtgccgggattacaggcatgagccaccgcgcccagcctcattgaaaatttacttttcaataccagactgcagagttctttggggcagagacactctgtcagtgtgctctttttccaaagtatctcctgctatcagtttttccccttgaaccaagtcttcctaacaaatttgttgtgtcattgctttctagggaactggaccagcaaatgtggcctttagtggttaatctcatctgtgccaaaatttagttgctaccagagtgaaatttggtgtgtaaataatgttcagaggaaatgtggttggagtgtagtaacttgaatagtgtcgtgcatagaaaacagctcattctgagtgaaactgtttatgtccaatcagttcctgagtcagcatcccactggttcgaaaatctgtaactaatctggtaatgtccttaatttcctcctgcctgtcagtgttccaaagtgttcatctagttttcttttatctttataataattactaccatctttgaaagttctgtttaagaaatgcttaatgggcaatgcgggttagtaactccagggtctgcgtggcctgggcaggtgagatggacaactgcctcattacagaagctttttatcatcaaactagtaagttttgtggagggcaagctatatagattgttgatgagtggttagaaaatcatctggaatttagccagttgagaagctacagaagtttctatttttttttacagtggatgaactgttttgctttttctgataaagccactaggtatatctaaataacaacctcgggctggacgtggtgccttatgcctgtatatgtgaacactgggaggcggaggcgggtggatcatgaggtcaagagtttgagaccagcctggccaacacagtgaaaccccgtctctactaagaatacaaattttttttgttttgagacagagtctcgctctgtcgcccaggctggagtgcaatggctcgatctcagctcactgcaacctccaccttctgggttcaagcgattctcctgcctcagcctcccgagtagctgggaatacaggcgcgtgtcaacacacccggcttaagtttttgtatttttagtagagacggggtgtcaccgtgttacccaagatggtctccatctcctgaccttgtgatccaccagcctttgcctcccaaagtgctgggattacaggcataagccaccgcgcccagcctaacaatacaaaatttagctaggcatggtggcacgtgcctgttatcacagctactcgggaggctgaggcaggagaaacgcttgaacacaggaggcagaagttgcggtaagccgagatcgtgccac

SEQ ID NO: 32 - protein, 524 aa, homo sapiens
MADDIDIEAMLEAPYKKDENKLSSANGHEERSKKRKKSKSRSRSHERKRSKSKERKRSRDRERKKSKSRERKRSRSKERRRSRSRSRDRRFRGRYRSPYSGPKFNSAIRGKIGLPHSIKLSRRRSRSKSPFRKDKSPVREPIDNLTPEERDARTVFCMQLAARIRPRDLEEFFSTVGKVRDVRMISDRNSRRSKGIAYVEFVDVSSVPLAIGLTGQRVLGVPIIVQASQAEKNRAAAMANNLQKGSAGPMRLYVGSLHFNITEDMLRGIFEPFGRIESIQLMMDSETGRSKGYGFITFSDSECAKKALEQLNGFELAGRPMKVGHVTERTDASSASSFLDSDELERTGIDLGTTGRLQLMARLAEGTGLQIPPAAQQALQMSGSLAFGAVADLQTRLSQQTEASALAAAASVQPLATQCFQLSNMFNPQTEEEVGWDTEIKDDVIEECNKHGGVIHIYVDKNSAQGNVYVKCPSIAAAIAAVNALHGRWFAGKMITAAYVPLPTYHNLFPDSMTATQLLVPSRR

(19) United States
(12) Patent Application Publication
(10) Pub. No.: US 2021/0251955 A1
(43) Pub. Date: Aug. 19, 2021

(54) PREVENTION OF AGE RELATED CLONAL HEMATOPOIESIS AND DISEASES ASSOCIATED THEREWITH

(71) Applicants: Yeda Research and Development Co. Ltd., Rehovot (IL); Memorial Sloan Kettering Cancer Center, New York, NY (US)

(72) Inventors: Liran SHLUSH, Herzeliya (IL); Omar ABDEL-WAHAB, New York, NY (US)

(73) Assignees: Yeda Research and Development Co. Ltd., Rehovot (IL); Memorial Sloan Kettering Cancer Center, New York, NY (US)

(21) Appl. No.: 17/242,530

(22) Filed: Apr. 28, 2021

Related U.S. Application Data
(63) Continuation of application No. PCT/IL2019/051165, filed on Oct. 28, 2019.

(30) Foreign Application Priority Data
Oct. 28, 2018 (IL) 262658

(51) Int. Cl. A61K 31/40; A61K 31/4725; A61K 31/496; A61K 31/506; A61K 31/4178; A61K 31/34; A61K 31/167; A61K 31/454; A61K 31/445; A61K 31/4545; A61K 31/351; A61K 31/404; A61K 45/06; A61P 35/02

(52) U.S. Cl. CPC A61K 31/40; A61K 31/4725; A61K 31/496; A61K 31/506; A61K 31/4178; A61K 31/34; A61P 35/02; A61K 31/454; A61K 31/445; A61K 31/4545; A61K 31/351; A61K 31/404; A61K 45/06; A61K 31/167

(57) ABSTRACT

A method of preventing a hematopoietic disorder or malignancy in a high risk subject, the subject being positive for one or more mutation in a splicing factor, is disclosed. The method comprising administering to the subject an agent capable of inhibiting spliceosomal activity, with the proviso that said agent does not inhibit RBM39 activity.

### RELATED APPLICATIONS

This application is a Continuation of PCT Patent Application No. PCT/IL2019/051165 having International filing date of Oct. 28, 2019, which claims the benefit of priority of Israel Patent Application No. 262658 filed on Oct. 28, 2018. The contents of the above applications are all incorporated by reference as if fully set forth herein in their entirety.

### SEQUENCE LISTING STATEMENT

The ASCII file, entitled 86876SequenceListing.txt, created on Apr. 28, 2021, comprising 110,606 bytes, submitted concurrently with the filing of this application is incorporated herein by reference.

### FIELD AND BACKGROUND OF THE INVENTION

The present invention, in some embodiments thereof, relates to the prevention of leukemia in high risk subjects carrying spliceosome machinery mutations, and, more particularly, but not exclusively, to the prevention of leukemia by the use of agents capable of inhibiting spliceosomal activity.

Acute myeloid leukemia (AML) and myelodysplastic syndromes (MDS) are hematological malignancies in great need for improved outcomes. Recent studies have changed the understanding of the pathogenesis of these disorders with the discovery that pre-leukemic hematopoietic stem and progenitor cells (preLHSPCs) carrying pre-leukemic mutations (pLMs) are the cells of origin in AML and MDS. preLHSPCs acquire leukemia-related mutations years before diagnosis and maintain almost normal function for years before transformation to overt disease. While pLMs can be found among individuals destined to develop AML and MDS, they are also present in 20-30% of healthy individuals, the majority of who will not develop AML/MDS in their lifetime. The presence of pLMs with no overt disease is termed age related clonal hematopoiesis (ARCH).

The role of ARCH somatic events in the early stages of AML was recently studied [Abelson et. al, Nature (2018) 559: 400-404 (2018)]. To understand whether specific mutations can predict AML years before diagnosis, peripheral blood (PB) was taken on average seven years before AML diagnosis, was sequenced and ARCH events were compared to those found in aged matched controls. While it was identified that the most commonly mutated genes in both pre-AML cases and controls were DNMT3a and TET2 mutations, it was surprisingly uncovered that spliceosome machinery mutations (SMMs) were almost exclusive to pre-AML. Nearly every individual carrying SMMs at the age of 50-60 developed AML. SMMs tended to occur at younger age among pre-AMLs versus controls. SMMs in overt MDS/AML mostly affect SF3B1, SRSF2, and U2AF1 at specific “hotspot” residues with mutations in SRSF2 and U2AF1 associated with adverse outcome in overt MDS/AML [Yoshida, K. et al. Nature (2011) 478: 64-69; Papaemmanuil, E. et al. N Engl J Med (2011) 365: 1384-1395; Graubert, T. A. et al. Nat Genet (2012) 44: 53-57]. In the pre-leukemic setting, SRSF2 mutations at codon P95 and in U2AF1 at codons S34 and Q157, were found predominantly in pre-AML cases. Carriers of U2AF1 mutations all developed AML.

SMMs in SF3B1, SRSF2, and U2AF1 are consistently heterozygous and occur as point mutations at highly restricted residues, suggesting that these are oncogenic change-of-function alterations. Consistent with this, transcriptomic analysis of cells bearing each of these alterations have identified that mutations in each of these factors alter splicing in a manner distinct from loss-of-function. For example, mutations affecting SRSF2, an auxiliary splicing factor that binds to exonic splicing enhancers to promote splicing, alter its RNA binding preferences in a sequence-specific manner and thereby alter the efficiency of exon inclusion. Mutations affecting U2AF1, the small subunit of the U2AF heterodimer which binds the AG dinucleotide at the 3′ss, promote or repress 3′ss based on sequences flanking the AG dinucleotide.

The effects of SF3B1, U2AF1, and SRSF2 mutations on splicing mechanisms are distinct, however, it is unclear why mutations affecting these three factors are mutually exclusive with one another. Evaluation of mice with inducible expression of mutations in Srsf2 or Sf3b1, alone or together, definitively demonstrated that each mutation affects RNA splicing in a distinct manner. Nonetheless, these two mutations were not tolerated when co-expressed in the same cell. Similarly, expression of mutant Srsf2 in a hemizgous)(Srsf2^{P95H/KO}) or homozygous (Srsf2^{P95H/P95H}) state resulted in a complete failure of hematopoiesis. Together, these data indicate that cells bearing SMMs are intolerant of further genetic perturbations to the splicing process [Kim et al., Cancer Cell (2015) 27(5): 617-630].

As SMM cells are dependent on expression of the remaining WT allele and co-existing spliceosomal gene mutations are synthetic lethal, it was further tested whether cells expressing SMMs might be sensitive to compounds that impair spliceosome function. Various compounds which impair spliceosome function were tested for AML treatment, including the drugs E7107 and H3B-8800, which bind SF3B1 and disrupt activity of the U2 snRNP component of the spliceosome. The anti-leukemic effects of E7107 was illustrated in isogenic MLL-AF9 murine AML models, with or without SRSF2 mutations, wherein it was identified that spliceosomal-mutant AML are differentially sensitive to inhibition of splicing in vivo over isogenic spliceosomal-WT counterparts [Lee et al. Nat Med (2016) 22: 672-678]. In AML patient-derived xenograft (PDX) models, with or without SF-mutations, it was illustrated that spliceosomal-mutant leukemias had a greater reduction in human leukemic cell burden in response to E7107 than their WT counterparts, however complete eradication could not be achieved [Lee et al., Nat Med (2016), supra]. An orally bioavailable analogue of E7107, termed H3B-8800, also induced dose-dependent reductions in splicing fidelity and exhibited preferential effects in SF3B1 as well as SRSF2-mutant AML and chronic myelomonocytic leukemia (CMML) [Seiler et al., Nature Medicine (2018) 24(4):497-504].

Additional Background Art Includes:

U.S. Patent Application no. 20150025017 discloses compositions and methods for treating cancer with antagonists of one or more spliceosome proteins PHF5A, U2AF1, or DDX1. Such spliceosome inhibitors include sudemycin, spliceostatin, FR901464, pladienolide, E7107, herboxidiene and meayamycin.

U.S. Patent Application no. 20140364439 discloses treatment of chronic lymphocytic leukemia (CLL) by administration of a compound that modulates SF3B1, e.g. spliceostatin, E7107, or pladienolide.

U.S. Patent Application no. 20160271149 discloses therapeutic compounds that suppress protein arginine methyltransferase activity to reduce tumor growth.

U.S. Patent Application no. 20180140578 discloses methods for treating cancer in a subject, wherein the subject displays a mutation in a splicing factor (i.e. U2AF1, SF3B1, SRSF2, and ZRSR2) and/or has an increased amount of DCAF15 compared to a control. Treatment is carried out by inhibiting an activity of RBM39 in the subject, such as by the use of an aryl sulfonamide (e.g. indisulam, tasisulam, chloroquinoxaline sulfonamide).

### SUMMARY OF THE INVENTION

According to an aspect of some embodiments of the present invention there is provided a method of preventing a hematopoietic disorder or malignancy in a high risk subject, wherein the subject is positive for one or more mutation in a splicing factor, the method comprising administering to the subject an agent capable of inhibiting spliceosomal activity, with the proviso that the agent does not inhibit RBM39 activity.

According to an aspect of some embodiments of the present invention there is provided an agent capable of inhibiting spliceosomal activity, with the proviso that the agent does not inhibit RBM39 activity, for use in preventing a hematopoietic disorder or malignancy in a high risk subject, wherein the subject is positive for one or more mutation in a splicing factor.

According to some embodiments of the invention, the agent capable of inhibiting spliceosomal activity is an agent capable of inhibiting a protein arginine methyltransferase (PRMT) as set forth in EC numbers 2.1.1.319, 2.1.1.320 or 2.1.1.321.

According to some embodiments of the invention, the agent capable of inhibiting spliceosomal activity is a splicing inhibitor.

According to some embodiments of the invention, the agent capable of inhibiting spliceosomal activity is a proteasomal degradation compound.

According to some embodiments of the invention, the PRMT is selected from the group consisting of a protein arginine methyltransferase 1 (PRMT1), a protein arginine methyltransferase 3 (PRMT3), a protein arginine methyltransferase 4 (PRMT4), a protein arginine methyl transferase 5 (PRMT5), a protein arginine methyltransferase 6 (PRMT6) and a protein arginine methyltransferase 9 (PRMT9).

According to some embodiments of the invention, the agent capable of inhibiting the PRMT is a polypeptide, a polynucleotide, or a small molecule.

According to some embodiments of the invention, the agent is a type I PRMT inhibitor MS-023 dihydrochloride, or a derivative or analog thereof.

According to some embodiments of the invention, the PRMT comprises PRMT5, the agent comprises GSK591 dihydrochloride or GSK3326595, or a derivative or analog thereof.

According to some embodiments of the invention, the PRMT comprises PRMT1, the agent comprises C-21, Furamidine dihydrochloride or TC-E 5003, or a derivative or analog thereof.

According to some embodiments of the invention, the PRMT comprises PRMT3, the agent comprises SGC707 or UNC2327, or a derivative or analog thereof.

According to some embodiments of the invention, the PRMT comprises PRMT4, the agent comprises MS049 oxalate salt or TP064, or a derivative or analog thereof.

According to some embodiments of the invention, the PRMT comprises PRMT6, the agent comprises MS049 oxalate salt, or a derivative or analog thereof.

According to some embodiments of the invention, the splicing inhibitor is a polypeptide, a polynucleotide, or a small molecule.

According to some embodiments of the invention, the splicing inhibitor is selected from the group consisting of a Sudemycin, a Spliceostatin, a FR901464, a Pladienolide, a Herboxidiene, a Meayamycin, an Isoginkgetin, a Madrasin, a Tetrocarcin, a N-palmitoyl-L-leucine, a Psoromic acid, a Clotrimazole, a NSC635326, a Napthazarin, an Erythromycin, a SAHA, a Garcinol, an Okadaic acid, a NB-506, a Ubistatin, a G5, or a derivative or analog thereof.

According to some embodiments of the invention, the splicing inhibitor is selected from the group consisting of a E7107, H3B-8800, FD-895, GEX1Q1-5, RQN-18690, NSC659999, BN82865, NSC95397, tetracycline, streptomycin, splitomicin, tautomycin, microcystin, siospyrin, chlorhexidine, or a derivative or analog thereof.

According to some embodiments of the invention, the proteasomal degradation compound targets a spliceosome associated protein selected from the group consisting of a core member of the SF3b complex, a U2AF complex, or a PRMT enzyme and a RNA binding protein.

According to some embodiments of the invention, the proteasomal degradation compound targets a spliceosome associated protein selected from the group consisting of SF3B1, SF3B2, SF3B3, PHF5a, U2AF1, U2AF2, PRMT5, PRMT1, PRMT2, PRMT3, PRMT4, PRMT6, PRMT8, SUPT6H, hnRNPH, and SRSF10.

According to some embodiments of the invention, the mutation is in a splicing factor selected from the group consisting of U2AF1, SF3B1, SRSF2, and ZRSR2.

According to some embodiments of the invention, the mutation is a point mutation.

According to some embodiments of the invention, the point mutation is an insertion, a deletion or a substitution.

According to some embodiments of the invention, the mutation is a mutation in S34 or Q157 in the U2AF1 polypeptide.

According to some embodiments of the invention, the mutation is a R625L, a N626H, a K700E, a G740E, a K741N, a Q903R, a E622D, a R625G, a Q659R, a H662Q, a H662D, a K666Q, a K666E, a K666N, a K666T, a K666R or a G742D mutation in the SF3B1 polypeptide.

According to some embodiments of the invention, the mutation is a mutation in P95 in the SRSF2 polypeptide.

According to some embodiments of the invention, the mutation is detected in pre-leukemic hematopoietic stem and progenitor cells.

According to some embodiments of the invention, the mutation is detected in a biological sample of the subject.

According to some embodiments of the invention, the hematopoietic disorder or malignancy is a leukemia.

According to some embodiments of the invention, the hematopoietic disorder or malignancy is a myelodysplastic syndrome (MDS).

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E illustrate the effects of PRMT inhibition on splicing. (FIG. 1A) Diagram of the role of PRMT5 and the type I PRMTs in splicing regulation. PRMTs methylate arginines on splicing proteins to promote spliceosome assembly and are required for proper splicing function. IC50 curves of spliceosomal mutant acute myeloid leukemia cells or their WT counterparts exposed to (FIG. 1B) the PRMT5 inhibitor GSK591 or (FIG. 1C) the PRMT1 inhibitor MS-023. Western blots of (FIG. 1D) symmetric dimethyl arginine (SDMA) or (FIG. 1E) asymmetric dimethyl arginine (ADMA) from the cells in (FIG. 1B) and (FIG. 1D) respectively.

FIG. 2 illustrates drug sensitivity of AML cells with SMMs or partial loss of PRMT5 to inhibitors of splicing, PRMTs, or LSD1. MLL-AF9 Srsf2^{WT/WT}; MLL-AF9 Srsf2^{P95H/WT} or MLL-AF9 Prmt5^{+/-} cells were treated for 7 days with the indicated compounds in 384 wells (5 increasing concentrations per compound). Viability at day 7 was scored by MTS assay and reported as the ratio over control treated cells (with equivalent dilution of DMSO). The experiment was conducted in biological triplicate, and each individual run was repeated in technical triplicate.

FIG. 3 illustrates that SMM AMLs are preferentially sensitive to inhibition of type I or type II PRMTs. Kaplan-Meier curves of recipient mice engrafted with MLL-AF9/Srsf2WT (black) or MLL-AF9/Srsf2 mutant (red) cells followed by treatment with the PRMT5 inhibitor GSK591 (left) or the type I PRMT inhibitor MS-023 (right).

FIGS. 4A-D illustrates that sulfonamides exhibit preferential effects on SMM cells through degradation of RBM39. (FIG. 4A) Sulfonamides bridge RBM39 to the CUL4-DDB1-DDA1-DCAF15 E3 ubiquitin ligase complex, leading to polyubiquitination and proteasomal degradation of RBM39. IC50 curves of the sulfonamide E7820 in isogenic (FIG. 4B) K562 and (FIG. 4C) NALM6 WT or mutant for SF3B1. (FIG. 4D) Western blots of RBM39 levels+1 mM of the sulfonamides Indusulam or E7820 from the cells in (FIG. 4C).

FIGS. 5A-D illustrate the preferential response of spliceosomal mutant hematopoietic cells to indisulam. (FIG. 5A) IC50 plots of isogenic K562 (left) and NALM-6 (right) cells to indisulam. These are cell with introduction of a spliceosomal gene mutation into the endogenous locus. (FIG. 5B) Western blots of RBM39 in the K562 cells from (FIG. 5A) at increasing doses of indisulam. (FIG. 5C) Log10 IC50 waterfall plots of response of AML cell lines with naturally occurring mutations in splicing factors to indisulam. Red bars represent cells with naturally occurring mutations in splicing factors. (FIG. 5D) Bar plot of number of differential splicing events across parental, SF3B1^{K700E/WT}, and SRSF2^{P95H/WT} K562 cells. The numbers above each bar indicate number of differentially spliced events.

### DESCRIPTION OF SPECIFIC EMBODIMENTS OF THE INVENTION

The present invention, in some embodiments thereof, relates to the prevention of leukemia in high risk subjects carrying spliceosome machinery mutations, and, more particularly, but not exclusively, to the prevention of leukemia by the use of agents capable of inhibiting spliceosomal activity.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Recent studies have changed the understanding of the pathogenesis of AML and MDS with the discovery that pre-leukemic hematopoietic stem and progenitor cells (preLHSPCs) carrying pre-leukemic mutations (pLMs) are the cells of origin in AML and MDS. preLHSPCs acquire leukemia-related mutations years before diagnosis and maintain almost normal function for years before transformation to overt disease. While pLMs can be found among individuals destined to develop AML and MDS, they are also present in 20-30% of healthy individuals, the majority of who will not develop AML/MDS in their lifetime. Identification of those individuals with age related clonal hematopoiesis (ARCH) who are at risk for AML/MDS represents a major challenge which could have important implications on the prevalence and therapy of MDS and AML.

While reducing the present invention to practice, the present inventors have uncovered means to treat patients with ARCH destined to develop pre-leukemia based on clinical parameters and have thus have uncovered means to prevent the development of pre-leukemia in these patients.

Specifically, the present inventors identified that the presence of spliceosome machinery mutations (SMMs) are highly predictive for pre-leukemia and can be used to identify and treat high-risk individuals with ARCH, at a time point before they have developed disease. Furthermore, the present inventors illustrated that splicing inhibitors, including spliceosome inhibitors (e.g. E7101, H3B-8800), compounds that suppress protein arginine methyltransferase activity (e.g. GSK591, GSK3326595) and/or proteasomal degradation compounds (e.g. sulfonamide drugs) can be used to target pre-leukemic cells carrying SMMs (e.g. carrying SRSF2 or U2AF1 hot spot mutations in their peripheral blood) in high-risk healthy individuals, thereby reducing clone size of cells carrying the SMMs, preventing their further outgrowth and preventing or delaying disease onset.

Thus, according to one aspect of the present invention there is provided a method of preventing a hematopoietic disorder or malignancy in a high risk subject, wherein the subject is positive for one or more mutation in a splicing factor, the method comprising administering to the subject an agent capable of inhibiting spliceosomal activity, with the proviso that the agent does not inhibit RBM39 activity.

According to one aspect of the present invention there is provided an agent capable of inhibiting spliceosomal activity, with the proviso that the agent does not inhibit RBM39 activity, for use in preventing a hematopoietic disorder or malignancy in a high risk subject, wherein the subject is positive for one or more mutation in a splicing factor.

According to a specific embodiment, the agent does not directly inhibit RBM39 activity

The phrase “directly inhibit” as used herein refers to an agent which interacts with RBM39 and inhibits its biological activity.

According to one embodiment, the agent does not directly promote RBM39 degradation.

The term “spliceosome” as used herein refers to the macromolecular complex responsible for removing intron sequences that interrupt many eukaryotic gene transcripts. The spliceosome is composed of five small nuclear ribonucleoproteins (snRNPs), known as U1, U2, U3, U4, U5 and U6, and more than 100 additional proteins.

As used herein, the term “splicing factor” refers to any of the proteins involved in the splicing of pre-mRNA on the spliceosome. Exemplary splicing factors include, but are not limited to, U2AF1 (U2 small nuclear RNA auxiliary factor 1, also known as U2AF35, having e.g. accession numbers NM_001025203.1 (SEQ ID NO: 1), NM_001025204.1 (SEQ ID NO: 2) or NM_006758.2 (SEQ ID NO: 3) (mRNA), or NP_006749.1 (SEQ ID NO: 4), NP_001020375.1 (SEQ ID NO: 5) or NP_001020374.1 (SEQ ID NO: 6) (protein)), a component of the U2 snRNP complex of the spliceosome; SF3B1 (splicing factor 3b subunit 1, also known as SF3B155 or SAP155, having e.g. accession numbers NM_001005526.2 (SEQ ID NO: 7), NM_001308824.1 (SEQ ID NO: 8) or NM_012433.3 (SEQ ID NO: 9) (mRNA), or NP_001295753.1 (SEQ ID NO: 10), NP_001005526.1 (SEQ ID NO: 11) or NP_036565.2 (SEQ ID NO: 12) (protein)); SRSF2 (serine and arginine rich splicing factor 2, also known as SC35 or SFRS2, having e.g. accession numbers NM_001195427.1 (SEQ ID NO: 13), NM_003016.4 (SEQ ID NO: 14) or XM_017024942.2 (SEQ ID NO: 15) (mRNA), or NP_003007.2 (SEQ ID NO: 16), NP_001182356.1 (SEQ ID NO: 17) or XP_016880431.1 (SEQ ID NO: 18) (protein)); and ZRSR2 (U2 small nuclear ribonucleoprotein auxiliary factor 35 kDa subunit-related protein 2, also known as URP, having e.g. accession numbers NM_005089.3 (SEQ ID NO: 19), XM_005274597.3 (SEQ ID NO: 20), XM_011545589.3 (SEQ ID NO: 21), XM_017029881.2 (SEQ ID NO: 22) or XM_017029882.2 (SEQ ID NO: 23) (mRNA), or XP_024308223.1 (SEQ ID NO: 24), XP_016885371.1 (SEQ ID NO: 25), XP_016885372.1 (SEQ ID NO: 26), NP_005080.1 (SEQ ID NO: 27), or XP_005274654.2 (SEQ ID NO: 28) (protein)).

In some embodiments, mutation/s in splicing factors, referred to as spliceosome machinery mutations (SMMs), can be used for the detection and prevention of hematopoietic disorders or malignancies. Such mutations typically affect spliceosomal gene products (e.g. spliceosomal proteins), result in defective cellular splicing machinery and consequently in defective RNA splicing of messenger RNA precursors (pre-mRNAs) into protein coding RNAs.

According to one embodiment, the mutation is a somatic mutation.

According to one embodiment, the mutation is a point mutation.

According to one embodiment, the point mutation is an insertion, a deletion or a substitution.

According to one embodiment, the mutation is of a single nucleotide (e.g. an insertion, deletion or substitution). Alternatively, the mutation may be of at least 2, 3, 4, 5, 10 or more nucleotides.

According to one embodiment, the mutation results in a missense mutation.

According to a specific embodiment, the mutation is a mutation in residues S34 or Q157 in the U2AF1 polypeptide. Thus, for example, the mutation may be a substitution from S to F or Y at amino acid 34 of a protein translated from the U2AF1 gene (e.g. as set forth in SEQ ID NOs: 4 and 6). According to another example, the mutation may be a substitution from Q to P or R at amino acid 157 of a protein translated from the U2AF1 gene (e.g. as set forth in SEQ ID NOs: 4 and 6).

According to a specific embodiment, the mutation is a mutation in residues R625, N626, K700, G740, K741, Q903, E622, R625, Q659, H662, K666 or G742 in the SF3B1 polypeptide. Thus, for example, the mutation may be a substitution from R to L at amino acid 625 (e.g. as set forth in SEQ ID NO: 12); a substitution from N to H at amino acid 626 (e.g. as set forth in SEQ ID NO: 12); a substitution from H to Q or D at amino acid 662 (e.g. as set forth in SEQ ID NO: 12); a substitution from K to E at amino acid 700 (e.g. as set forth in SEQ ID NO: 12); a substitution from G to E at amino acid 740 (e.g. as set forth in SEQ ID NO: 12); a substitution from K to N at amino acid 741 (e.g. as set forth in SEQ ID NO: 12); a substitution from Q to R at amino acid 903 (e.g. as set forth in SEQ ID NO: 12); a substitution from E to D at amino acid 622 (e.g. as set forth in SEQ ID NO: 12); a substitution from R to G at amino acid 625 (e.g. as set forth in SEQ ID NO: 12); a substitution from Q to R at amino acid 659 (e.g. as set forth in SEQ ID NO: 12); a substitution from K to N, T, E, R or Q at amino acid 666 (e.g. as set forth in SEQ ID NO: 12); a substitution from G to D at amino acid 742 (e.g. as set forth in SEQ ID NO: 12), of a protein translated from the SF3B1 gene.

According to a specific embodiment, the mutation is a mutation in residue P95 in the SRSF2 polypeptide. Thus, for example, the mutation may be a substitution from P to H, L, or R at amino acid 95 of a protein translated from the SRSF2 gene (e.g. as set forth in SEQ ID NOs: 16-18).

In some embodiments, the subject displays an increased level of DCAF15 as compared to a healthy subject

The term “healthy subject” as used herein refers to a subject who does not have the mutation in a splicing factor, has not been diagnosed with a hematopoietic disorder or malignantly, does not suffer from the symptoms of a hematopoietic disorder or malignantly, and is not at high risk of developing a hematopoietic disorder or malignancy (as discussed below).

As used herein, the term “DCAF15” refers to the DDB1 And CUL4 Associated Factor 15 from Homo sapiens, having accession number NP_612362.2 (SEQ ID NO: 30) (protein) or NM_138353.3 (SEQ ID NO: 29) (mRNA).

In a specific embodiment, the subject displays a mutation in a splicing factor (e.g. U2AF1, SF3B1, SRSF2, and ZRSR2) has an increased level of DCAF15 as compared to a healthy subject.

Any method known in the art may be used to detect a mutation. For example, chromosomal and DNA staining methods may be carried out including, but not limited to:

Fluorescence in situ Hybridization (FISH) analysis on interphase chromosomes as taught e.g. Quijada-Alamo M. et al. J Hematol Oncol. 2017; 10: 83);

PRINS analysis employed in the detection of gene deletion (Tharapel S A and Kadandale J S, 2002. Am. J. Med. Genet. 107: 123-126), in determination of fetal sex (Orsetti, B., et al., 1998. Prenat. Diagn. 18: 1014-1022), and in identification of chromosomal aneuploidy (Mennicke, K. et al., 2003. Fetal Diagn. Ther. 18: 114-121);

High-resolution multicolor banding (MCB) on interphase chromosomes, as described in detail by Lemke et al. (Am. J. Hum. Genet. 71: 1051-1059, 2002), which uses YAC/BAC and region-specific microdissection DNA libraries as DNA probes for interphase chromosomes;

Quantitative FISH (Q-FISH), by which chromosomal abnormalities are detected by measuring variations in fluorescence intensity of specific probes. Q-FISH can be performed using Peptide Nucleic Acid (PNA) oligonucleotide probes as previously described (Pellestor F and Paulasova P, 2004; Chromosoma 112: 375-380). Alternatively, Q-FISH can be performed by co-hybridizing whole chromosome painting probes (e.g., for chromosomes 21 and 22) on interphase nuclei as described in Truong K et al, 2003, Prenat. Diagn. 23: 146-51.

Additionally or alternatively, to determine sequence alterations, e.g., a single nucleotide polymorphism (SNP), in the splicing factor gene, a variety of methods may be employed including, but not limited to:

Restriction fragment length polymorphism (RFLP)—This method uses a change in a single nucleotide (the SNP nucleotide) which modifies a recognition site for a restriction enzyme resulting in the creation or destruction of an RFLP. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Sequencing analysis—The isolated DNA is subjected to automated dideoxy terminator sequencing reactions using a dye-terminator (unlabeled primer and labeled di-deoxy nucleotides) or a dye-primer (labeled primers and unlabeled di-deoxy nucleotides) cycle sequencing protocols. For the dye-terminator reaction, a PCR reaction is performed using unlabeled PCR primers followed by a sequencing reaction in the presence of one of the primers, deoxynucleotides and labeled di-deoxy nucleotide mix. For the dye-primer reaction, a PCR reaction is performed using PCR primers conjugated to a universal or reverse primers (one at each direction) followed by a sequencing reaction in the presence of four separate mixes (correspond to the A, G, C, T nucleotides) each containing a labeled primer specific the universal or reverse sequence and the corresponding unlabeled di-deoxy nucleotides.

Microsequencing analysis—This analysis can be effected by conducting microsequencing reactions on specific regions of the splicing factor gene which may be obtained by amplification reaction (PCR) such as mentioned hereinabove. Microsequencing protocol are described e.g. by Nyren et al. (1993) Anal Biochem 208(1):171-175 and Pastinen et al. (1997) Genome Research 7:606-614.

Mismatch detection assays based on polymerases and ligases—The “Oligonucleotide Ligation Assay” (OLA) uses two oligonucleotides which are designed to be capable of hybridizing to abutting sequences of a single strand of target molecules. One of the oligonucleotides is biotinylated, and the other is detectably labeled. OLA is capable of detecting single nucleotide polymorphisms and may be advantageously combined with PCR as described by Nickerson et al. (1990) Proc. Natl. Acad. Sci. U.S.A. 87:8923-8927.

Ligase/Polymerase-mediated Genetic Bit Analysis™—another method for determining the identity of a nucleotide at a preselected site in a nucleic acid molecule (discussed in WO 95/21271).

Hybridization Assay Methods—Hybridization based assays which allow the detection of single base alterations rely on the use of oligonucleotide which can be 10, 15, 20, or 30 to 100 nucleotides long. U.S. Pat. No. 5,451,503 provides several examples of oligonucleotide configurations which can be utilized to detect SNPs in template DNA or RNA.

Hybridization to oligonucleotide arrays—The chip/array technology as described e.g. for screening of mutations in the BRCA1 gene, in S. cerevisiae mutant strains, and in the protease gene of HIV-1 virus [see Hacia et al., (1996) Nat Genet 1996; 14(4):441-447; Shoemaker et al., (1996) Nat Genet 1996; 14(4):450-456; Kozal et al., (1996) Nat Med 1996; 2(7):753-759].

Integrated Systems—Another technique which may be used to analyze sequence alterations includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Allele specific oligonucleotide (ASO)—In this method an allele-specific oligonucleotides (ASOs) is designed to hybridize in proximity to the polymorphic nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983).

Additional sequencing methods which may be employed include, e.g. Pyrosequencing™ analysis (Pyrosequencing, Inc. Westborough, Mass., USA), Acycloprime™ analysis (Perkin Elmer, Boston, Mass., USA), the sequencing methods described in U.S. Patent Application No. 20150025017 (incorporated herein by reference), RNA sequencing (RNA-seq), microarray analysis, serial analysis of gene expression (SAGE), MassARRAY® technique, or any combination thereof.

Additionally or alternatively, detection of a mutation may be carried out at the polypeptide level, for example, using amino acid sequence analysis of the peptide, such as gas-phase sequencer, the Edman method utilizing immunological specificity reactions such as Enzyme Immunoassay (EIA)/Enzyme-Linked Immunosorbent Assay (ELISA), or by mass spectrometry, such as Q-TOF/MS method.

As used herein, the term “preventing” refers to keeping a disease, disorder or condition from occurring in a subject who may be at risk for the disease or disorder, but has not yet been diagnosed as having the disease or disorder, e.g. a hematopoietic disorder or malignancy (e.g., a leukemia or MDS).

As used herein, the term “subject” or “subject in need thereof” refers to animals, including mammals, preferably human beings, at any age or any gender that are at risk of developing a pathology, e.g. a hematopoietic disorder or malignancy (e.g., a leukemia or MDS).

According to one embodiment, the subject is undergoing a routine well-being check-up.

According to one embodiment, the subject is under 70 years old, under 65 years old, under 60 years old, under 55 years old, under 50 years old, under 45 years old, under 40 years old, under 35 years old, under 30 years old, under 25 years old or under 20 years old.

According to one embodiment, the subject is at risk of developing a hematopoietic disorder or malignancy (e.g., a human who is genetically or otherwise predisposed to developing a hematopoietic disorder or malignancy) and who has not been diagnosed with the hematopoietic disorder or malignancy (e.g. leukemia or MDS).

According to a specific embodiment, the subject has at least one mutation in a splicing factor but does not suffer from symptoms of the hematopoietic malignancy, e.g. assembly of any of the following symptoms: larger cell clones (measured by peripheral blood variant allele fraction (PB-VAF)), more than one ARCH defining event, increased red cell distribution width (RDW), reduced monocyte cell counts, reduced platelet cell counts, reduced red blood cell counts, reduced white blood cell counts, reduced hemoglobin levels, reduced cholesterol levels, prolonged fever, enlarged lymph nodes and/or spleen.

As used herein, a “high risk subject” is a subject who is likely to develop a hematopoietic disorder or malignancy (e.g., a leukemia or MDS) due to one or more so-called risk factors, which are measurable parameters that correlate with development of the hematopoietic disorder or malignancy, such as described herein. A subject having one or more of these risk factors has a higher probability of developing the hematopoietic disorder or malignancy as compared to an individual without these risk factor(s).

According to one embodiment, the subject has not been diagnosed as having the disease or disorder, e.g. hematopoietic disorder or malignancy, e.g., a leukemia or MDS.

According to one embodiment, the subject is positive for one or more mutation in a splicing factor (as discussed above).

As used herein, the term “positive” refers to a genome of the subject that displays at least one mutation in a splicing factor as determined by any method known in the art (discussed in detail hereinabove).

Additional risk factors may include, for example, age, gender, race, diet, weight, history of a previous disease, presence of a precursor disease (e.g. pre-leukemia), genetic (e.g., hereditary) considerations, and environmental exposure (e.g. radiation or chemical exposure). In some embodiments, a subject at high risk of developing a hematopoietic disorder or malignancy (e.g., a leukemia or MDS) include, for example, a subject whose relatives have experienced this disease, and whose risk is determined by analysis of genetic or biochemical marker/s. Such subjects may be identified by the presence of certain genetic aberrations, as discussed in detail below.

In some embodiments, a subject at high risk of developing a hematopoietic disorder or malignancy (e.g., a leukemia or MDS) if they exhibit any of, but not limited to, larger cell clones (measured by peripheral blood variant allele fraction (PB-VAF)), more than one ARCH defining event, increased red cell distribution width (RDW), reduced monocyte cell counts, reduced platelet cell counts, reduced red blood cell counts, reduced white blood cell counts, reduced hemoglobin levels, reduced cholesterol levels, prolonged fever, enlarged lymph nodes and/or spleen, or any combination thereof, as compared to subject not at high risk of developing a hematopoietic disorder or malignancy (e.g., a leukemia or MDS), e.g. a healthy subject.

Determination of risk factors may be carried out by any person of skill in the art, such as by the use of questionnaires, by physical examination and using standard blood tests (e.g. CBC).

According to one embodiment, the high risk subject has a single SMM mutation.

According to one embodiment, the high risk subject has 2, 3, 4, 5 or more SMM mutations (as discussed above).

According to one embodiment, the subject has a combination of risk factors (e.g. a SMM mutation along with any of the risk factors discussed above, e.g. age, gender, race, cell counts, hemoglobin levels, etc.).

Thus, it is understood that methods of prevention as detailed herein may, in some instances, employ selecting a subject who is at high risk by detecting the presence or absence of one or more SMM mutation, e.g. a U2AF1 and SRSF2 mutation, or any combination thereof. Additionally, the methods of the invention may, in some instances, employ selecting a subject who is at high risk by assessing any of the above described risk factors (e.g. age, gender, race, cell counts, hemoglobin levels, or any combination thereof).

According to one embodiment, the mutation and/or other risk factor is detected in hematopoietic stem and progenitor cells.

According to one embodiment, the mutation and/or other risk factor is detected in pre-leukemic hematopoietic stem and progenitor cells.

According to one embodiment, the mutation and/or other risk factor is detected in a biological sample of the subject.

As used herein “biological sample” refers to a sample of tissue or fluid isolated from a subject, including but not limited to, whole blood, plasma, serum, blood cells, spinal fluid, lymph fluid, the external sections of the skin, respiratory, intestinal, and genitourinary tracts, tears, saliva, sputum, milk, tumors, cysts, neuronal tissues, organs, and also samples of in vivo cell culture constituents.

According to a specific embodiment, the biological sample comprises pre-leukemic hematopoietic stem and progenitor cells.

Numerous well known tissue or fluid collection methods can be utilized to collect the biological sample from the subject in order to determine the presence of a mutation. Collections methods include, but are not limited to, fine needle biopsy, needle biopsy, core needle biopsy and surgical biopsy (e.g., brain biopsy), buccal smear and lavage. Regardless of the procedure employed, once a biopsy/sample is obtained the level of the variant can be determined and a selection can thus be made.

A number of diseases and conditions, which involve a mutation in a splicing factor, can be prevented using the present teachings. The most prevalent conditions involving a mutation in a splicing factor are hematopoietic disorders or malignancies.

The term “hematopoietic disorder” refers to any blood disorder including but not limited to hematopoietic malignancy, hemoglobinopathy, and immunodeficiency.

The term “hematopoietic malignancy” (also named hematological malignancies) as used herein refers to any blood cell cancer, characterized by uncontrolled, abnormal growth of blood cells. The term “hematopoietic malignancy” includes but is not limited to, leukemia, myelodysplastic syndrome (MDS), lymphoma, and plasma cell dyscrasia.

The term “leukemia” refers to a disease of the blood forming tissues characterized by an abnormal increase in the number of leukocytes in the tissues of the body with or without a corresponding increase of those in the circulating blood. Leukemia of the present invention includes lymphocytic (lymphoblastic) leukemia and myelogenous (myeloid or nonlymphocytic) leukemia. Exemplary types of leukemia include, but are not limited to, chronic lymphocytic leukemia, (CLL), chronic myelocytic leukemia (CML) [also known as chronic myelogenous leukemia (CML)], acute lymphoblastic leukemia (ALL) and acute myeloid leukemia (AML) [also known as acute myelogenous leukemia (AML), acute nonlymphocytic leukemia (ANLL) and acute myeloblastic leukemia (AML)].

The term “acute leukemia” means a disease that is characterized by a rapid increase in the numbers of immature blood cells that transform into malignant cells, rapid progression and accumulation of the malignant cells, which spill into the bloodstream and spread to other organs of the body.

The term “chronic leukemia” means a disease that is characterized by the excessive build up of relatively mature, but abnormal, white blood cells.

According to one embodiment, the leukemia is an acute myeloid leukemia (AML).

The term “myelodysplastic syndrome” or “MDS” refers to a condition in which the bone marrow shows qualitative and quantitative changes suggestive of a pre-leukemic process, but having a chronic course that does not necessarily terminate as acute leukemia.

The term “lymphoma” refers to a malignant tumor of lymphoblasts derived from B lymphocytes. Exemplary types of lymphoma include, but are not limited to, Hodgkin lymphoma (HL), non-Hodgkin lymphoma (NHL), mature B-cell lymphoma, mature T-cell and natural killer cell lymphomas, and immunodeficiency-associated lymphoproliferative disorders.

The term “plasma cell dyscrasia” refers to plasmacytosis due to plasma cell proliferation. Exemplary types of plasma cell dyscrasia include, but are not limited to, multiple myeloma (MM) and plasma cell leukemia (PCL).

The term “hemoglobinopathy” as used herein refers to disorders involving the oxygen-carrying component of blood known as hemoglobin. Exemplary types of hemoglobinopathy include, but are not limited to, sickle cell anemia, Fanconi anemia and thalassemia.

The term “immunodeficiency” as used herein refers to the inability to mount a normal immune response. The term “immunodeficiency” encompasses both inherited (genetic) and acquired immunodeficiencies. Exemplary types of immunodeficiencies include, but are not limited to, severe combined immunodeficiency (SCID), X-linked agammaglobulinemia (XLA), common variable immunodeficiency (CVID), immune-complex diseases (e.g. viral hepatitis) and AIDS.

As mentioned hereinabove, the methods of the present invention are performed by administering to a subject in need thereof a therapeutically effective amount of an agent capable of inhibiting spliceosomal activity, with the proviso that the agent does not inhibit RBM39 activity. Such agents are lethal to cells already comprising a spliceosome machinery mutation (e.g. pre-leukemic cells) as these cells are dependent on expression of the remaining wild type allele.

The term “inhibiting spliceosomal activity” refers to decreasing splicing activity in a cell by at least about 10%, 20%, 30%, 40%, 50%, 60%, 70%, 80%, 90%, 95%, 99% or 100% as compared to a cell not contacted with the agent.

Decreasing splicing activity can be effected by downregulating the expression, assembly or activity of a spliceosome protein (e.g. splicing factor). Accordingly, downregulating a spliceosome protein (e.g. splicing factor) can be effected at the genomic level (e.g. by homologous recombination and site specific endonucleases) and/or the transcript level using a variety of molecules which interfere with transcription and/or translation (e.g., RNA silencing agents) and/or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Downregulation of expression may be either transient or permanent.

According to specific embodiments, downregulating expression refers to the absence of mRNA and/or protein, as detected by RT-PCR or Western blot, respectively.

According to other specific embodiments downregulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of inhibiting spliceosomal activity by downregulating expression of spliceosome proteins (e.g. splicing factors) are described in details hereinbelow.

According to one embodiment, the agent directly downregulates an activity or expression of a splicing factor. The term “directly” means that the agent acts upon and/or directly interacts with the splicing factor nucleic acid sequence or protein and not on a co-factor, an upstream activator or downstream effector of the splicing factor. Such an agent typically blocks splicing.

According to one embodiment, inhibition of spliceosomal activity is effected by a splicing inhibitor.

According to one embodiment, the splicing inhibitor interferes with spliceosome assembly such that no splicing complex formation occurs.

According to one embodiment, the splicing inhibitor interferes with acetylation state of a spliceosome protein.

According to one embodiment, the splicing inhibitor targets kinases and phosphatases linked to spliceosome activity.

According to one embodiment, the agent inhibits a splicing factor including, but not limited to, U2AF1, SF3B1, SRSF2, and ZRSR2.

Exemplary splicing inhibitors include, but are not limited to, Sudemycin, Spliceostatin, FR901464, Pladienolide, Herboxidiene, Meayamycin, Isoginkgetin, Madrasin, Tetrocarcin, N-palmitoyl-L-leucine, Psoromic acid, Clotrimazole, NSC635326, Napthazarin, Erythromycin, SAHA, Garcinol, Okadaic acid, NB-506, Ubistatin, G5, or a derivative or analog thereof.

According to a specific embodiment, the splicing inhibitors include, but are not limited to, E7107, H3B-8800, FD-895, GEX1Q1-5, RQN-18690, NSC659999, BN82865, NSC95397, tetracycline, streptomycin, splitomicin, tautomycin, microcystin, siospyrin, chlorhexidine, or a derivative or analog thereof.

Additional splicing inhibitors which can be used in accordance with the present teachings are disclosed in Kerstin A. Effenberger, Veronica K. Urabe, and Melissa S. Jurica, “Modulating splicing with small molecular inhibitors of the spliceosome”, Wiley Interdiscip Rev RNA. Author manuscript; PMC 2018 Mar. 1, incorporated herein by reference in its entirety.

According to one embodiment, the agent indirectly downregulates an activity or expression of a splicing factor. The term “indirectly” means that the agent acts upon a co-factor, an upstream activator or downstream effector of the splicing factor.

According to one embodiment, inhibition of spliceosomal activity is effected by perturbing splicing through proteasomal degradation.

According to a specific embodiment, the agent does not inhibit RBM39 activity.

According to one embodiment, the agent does not alter the biological activity of RBM39.

According to one embodiment, the agent does not lead to RBM39 ubiquitination as determined by In Vivo Polyubiquitination Assay.

According to one embodiment, the agent does not lead to RBM39 degradation, as determined by e.g. Western blot assay, C-terminal tagging of endogenous RBM39 assay, Auxin induced degradation of RBM39 assay and/or Immunoprecipitation of RBM39 Complex.

According to one embodiment, the agent does not lead to RBM39 ubiquitination and degradation.

The term “RBM39” refers to RNA binding motif protein 39. In some embodiments, the RBM39 is from Homo sapiens, having accession number NM_004902 (SEQ ID NO: 31) (mRNA) or NP_004893 (SEQ ID NO: 32) (protein).

According to one embodiment, the agent is not an aryl sulfonamide.

According to a specific embodiment, the agent is not indisulam.

According to a specific embodiment, the agent is not tasisulam.

According to a specific embodiment, the agent is not chloroquinoxaline sulfonamide (CQS).

According to one embodiment, the agent promotes degradation of a spliceosome protein comprising, but not limited to, core members of the SF3b complex (e.g. SF3B1, SF3B2, SF3B3, and PHF5a), U2AF complex (e.g. U2AF1, U2AF2), PRMT enzymes (e.g. PRMT5, PRMT1, PRMT2, PRMT3, PRMT4, PRMT6, and PRMT8) or RNA binding proteins (RBPs, e.g. SUPT6H, hnRNPH, and SRSF10).

According to a specific embodiment the agent is capable of proteasomal degradation of core members of the SF3b complex (e.g. SF3B1, SF3B2, SF3B3, and PHF5a), U2AF complex (e.g. U2AF1, U2AF2), PRMT enzymes (e.g. PRMT5, PRMT1, 2, 3, 4, 6, and 8) or RNA binding proteins (RBPs, e.g. SUPT6H, hnRNPH, and SRSF10) can be used in accordance with the present teachings.

According to one embodiment, inhibition of spliceosomal activity is effected by inhibition of a protein arginine methyltransferase (PRMT).

As used herein, “protein arginine methyltransferase” or “PRMT” refers to the family of proteins which regulate expression of a wide spectrum of target genes through their ability to catalyze symmetric or asymmetric methylation of histones and non-histone proteins.

Exemplary PRMTs include, but are not limited to, protein arginine methyltransferase 1 (PRMT1, e.g. as set forth in EC number 2.1.1.319), protein arginine methyltransferase 2 (PRMT2, e.g. as set forth in EC number 2.1.1.319), protein arginine methyltransferase 3 (PRMT3, e.g. as set forth in EC number 2.1.1.-), protein arginine methyltransferase 4 (PRMT4), protein arginine methyl transferase 5 (PRMT5, e.g. as set forth in EC number 2.1.1.320), protein arginine methyltransferase 6 (PRMT6, e.g. as set forth in EC number 2.1.1.319), protein arginine methyltransferase 7 (PRMT7, e.g. as set forth in EC number 2.1.1.321), protein arginine methyltransferase 8 (PRMT8, e.g. as set forth in EC number 2.1.1.-), and protein arginine methyltransferase 9 (PRMT9, also referred to as 4q31).

According to one embodiment, the agent inhibits a methyltransferase activity of the protein arginine methyltransferase.

According to a specific embodiment, the agent is a type I PRMT inhibitor MS-023 dihydrochloride, or a derivative or analog thereof.

According to a specific embodiment, when the PRMT comprises PRMT5, the agent comprises GSK591 dihydrochloride or GSK3326595, or a derivative or analog thereof.

According to a specific embodiment, when the PRMT comprises PRMT1, the agent comprises C-21, Furamidine dihydrochloride or TC-E 5003, or a derivative or analog thereof.

According to a specific embodiment, when the PRMT comprises PRMT3, the agent comprises SGC707 or UNC2327, or a derivative or analog thereof.

According to a specific embodiment, when the PRMT comprises PRMT4, the agent comprises MS049 oxalate salt or TP064, or a derivative or analog thereof.

According to a specific embodiment, when the PRMT comprises PRMT6, the agent comprises MS049 oxalate salt, or a derivative or analog thereof.

Additional PRMT inhibitors which can be used in accordance with the present teachings are disclosed in H. Ümit Kaniskan, Michael L. Martini, and Jian Jin, “Inhibitors of Protein Methyltransferases and Demethylases”, Chem. Rev. (2018) 118: 989-1068; and Hao Hu, Kun Qian, Meng-Chiao Ho, and Y. George Zheng “Small Molecule Inhibitors of Protein Arginine Methyltransferases”, Expert Opin Investig Drugs. (2016) 25(3): 335-358, both incorporated herein by reference in their entirety.

In addition to the agents discussed above, agents capable of downregulating a spliceosome protein (e.g. splicing factor) may be any molecule which binds to and/or cleaves the spliceosome protein (e.g. splicing factor). Such molecules can be small molecules, antagonists, or inhibitory peptides.

It will be appreciated that a non-functional analogue of at least a catalytic or binding portion of a spliceosome protein (e.g. splicing factor) can be also used as an agent which inhibits spliceosome activity.

Alternatively or additionally, small molecule or peptides can be used which interfere with a spliceosome protein (e.g. splicing factor) protein function (e.g., catalytic or interaction).

Another agent which can be used along with some embodiments of the invention to downregulate a spliceosome protein (e.g. splicing factor) is a molecule which prevents a spliceosome protein (e.g. splicing factor) activation or substrate binding.

Additional agents capable of inhibiting spliceosomal activity at the polypeptide level include antibodies, antibody fragments, and aptamers.

According to specific embodiments the agent capable of downregulating a spliceosome protein (e.g. splicing factor) is an antibody or antibody fragment capable of specifically binding the spliceosome protein (e.g. splicing factor). Preferably, the antibody specifically binds at least one epitope of a spliceosome protein (e.g. splicing factor). As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As the spliceosome protein (e.g. splicing factor) is localized intracellularly, an antibody or antibody fragment capable of specifically binding the spliceosome protein (e.g. splicing factor) is typically an intracellular antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Another agent which can be used along with some embodiments of the invention to downregulate a spliceosome protein (e.g. splicing factor) is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of a spliceosome protein (e.g. splicing factor) can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., splicing factor) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the spliceosome protein (e.g. splicing factor) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably includes the same nucleotide composition as the siRNAs but lack significant homology to the genome. Thus, a scrambled nucleotide sequence of the siRNA is preferably used, provided it does not display any significant homology to any other gene.

For example, suitable siRNAs directed against a spliceosome protein (e.g. splicing factor) can be the commercially obtained from Santa Cruz Biotechnology, Inc.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a spliceosome protein (e.g. splicing factor) can be effected using an antisense polynucleotide capable of specifically hybridizing with an mRNA transcript encoding spliceosome protein (e.g. splicing factor).

Design of antisense molecules which can be used to efficiently downregulate a spliceosome protein (e.g. splicing factor) must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Nucleic acid agents can also operate at the DNA level as summarized infra.

Downregulation of spliceosome protein (e.g. splicing factor) can also be achieved by inactivating the gene (e.g. splicing factor) via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., splicing factor) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. spliceosome protein (e.g. splicing factor) may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR.

Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site.

The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. Basically, the site specific recombinase system offers means for the removal of selection cassettes after homologous recombination. Transposases—As used herein, the term “transposase” refers to an enzyme that binds to the ends of a transposon and catalyzes the movement of the transposon to another part of the genome. As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

Inhibition of spliceosomal activity can be assessed using any method known in the art, such as using growth inhibition or cytotoxicity of cells in culture. Additionally or alternatively, in vitro assays measuring splicing of select endogenous gene transcripts can be carried out. Such methods are discussed in Kerstin A. Effenberger, Veronica K. Urabe, and Melissa S. Jurica, “Modulating splicing with small molecular inhibitors of the spliceosome”, Wiley Interdiscip Rev RNA. Author manuscript; PMC 2018 Mar. 1, incorporated herein by reference in its entirety.

The agents of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent capable of inhibiting spliceosomal activity accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agent capable of inhibiting spliceosomal activity) effective to prevent, alleviate or ameliorate symptoms of a disorder (e.g., leukemia or MDS) or prolong the survival of the subject being treated.

Determination of a therapeutically effective amount is well within the capability of those skilled in the art, especially in light of the detailed disclosure provided herein.

Animal models for pre-leukemia are described for example in Maggio et al., Yale J Biol Med. (1978) 51(4):469-76 and Cook et al., Cancer Metastasis Rev. (2013) June; 32(0): 63-76.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide pre-leukemic cells (e.g. hematopoietic stem and progenitor cells) levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be prevented, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another embodiment, in order to enhance prevention of the hematopoietic disorder or malignancy, the present invention further envisions administering to the subject an additional therapy which may benefit treatment. One of skill in the art is capable of making such a determination.

Thus, for example, the compositions described herein may be administered in conjunction with dietary supplements, hormonal therapy, targeted therapy, immunotherapy, chemotherapy, radiation therapy or surgical procedures. Such anti-cancer therapies and methods of utilizing same are well known to one of skill in the art.

It is expected that during the life of a patent maturing from this application many relevant agents capable of inhibiting spliceosomal activity will be developed and the scope of the term agents is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Cells

Samples from autologous stem cell transplantation were identified and sequenced for SMMs. These samples contained high frequency of human preL-HSPCs which could be studied in xenograft models.

Animals

PreL-HSPCs from carriers of SMMs were injected to the right femur of NSG mice or NSG-SGM3. The injected mice and control group were treated with a spliceosome inhibitor. Human engraftment was assessed after 8 weeks by harvesting bone marrow cells and estimating the fraction of human cells by anti-human CD45.

Inhibition of Protein Arginine Methyltransferases (PRMTs)

Isogenic murine Srsf2^{WT} and mutant (Srsf2^{P95H/WT}) MLL-AF9 AML cells were treated with several methyltransferase/demethylase inhibitors (chemical probe collection from the Structural Genomics Consortium Toronto), including a PRMT5 inhibitor (GSK591) and the pan-PRMT type I inhibitor (MS-023) in vitro. The SF3B1 inhibitor, E7107, was used in the same assays as a positive control.

Proteosomal Degradation of RBM39

The effects of indisulam and the sulfonamide analogue, E7820, were first examined across a panel of 18 genetically diverse AML cell lines.

In Vivo Drug Studies

Samples carrying SMMs capable of engrafting both myeloid and lymphoid cells (multi-lineage xenografting) which carry SMMs following engraftment were used. To test whether any treatment modulating SMMs is selectively targeting the preL-HSPCs, the total human engraftment was measured in treated and untreated control groups. In addition, variant allele fraction (VAF) of SMMs in human cells extracted from mice, were measured in treated versus non-treated mice. CD34⁺ cells (10,000-50,000) were injected intrafemorally (IF) to 5 treated mice and 5 untreated mice. On day 35 post-injection mice were treated with the following compounds: H3B-8800, GSK3326595, MS-023, E7820, or vehicle control. On day 56 following cells injection, mice were sacrificed. Other control experiments included the same experiments, using CD34⁺ cells derived from human cord blood and from elderly people with no recurrent pre-leukemic mutations. To provide evidence that targeting preL-HSPCs is more favorable than treating full-blown leukemia, AML samples with SMMs were also injected to NSG and SGM3 mice and leukemic engraftment (CD33⁺) was identified.

Administration of Spliceosome Modulator E7107 In Vitro and In Vivo

For all in vitro experiments E7107 was dissolved in DMSO. For drug sensitivity studies, cells were exposed to E7107 from a range of 10 μM to 0.05 nM. For in vivo administration, E7107 was dissolved in vehicle (10% ethanol and 4% Tween-80 in sterile PBS) and administered via I.V. injection at 4 mg/kg/day. For drug efficacy studies, randomization was done by conducting complete blood count (CBC) analysis prior to the start of drug administration and confirming that WBC count averages were equivalent in treatment and vehicle groups. All mice received 10 consecutive doses of E7107. No blinding was done in the in vivo drug studies or data analysis. For RNA-seq analysis in mouse MLL-AF9 leukemia model, 5 consecutive doses of E7107 were administered, and mice were sacrificed 3 hours after the last dose and BM Mac1⁺GFP⁺ cells were purified by flow cytometry.

RNA-Seq Read Mapping

All human and mouse samples were processed using the same pipeline.

Step 1: The reads were mapped to their respective genome assembly, using Bowtie v1.0.0 and RSEM v.1.2.4. The latter was internally modified to call Bowtie with -v 2, and was run on the gene annotation file with the parameters —bowtie-m 100 —bowtie-chunkmbs 500 —calc-ci —output-genome-bam.

Step 2: BAM files from step 1 were filtered to remove reads where (i) the alignment mapq score was 0, and (ii) the splice junction overhang was less than 6 nucleotides.

Step 3: All remaining unaligned reads were mapped to the splice junction annotation files using TopHat v2.0.8b called with the parameters —bowtie1 —read-mismatches 3 —read-edit-dist 2 —no-mixed —no-discordant —min-anchor-length 6 —splice-mismatches 0 —min-intron-length 10 —max-intron-length 1000000 —min-isoform-fraction 0.0 —no-novel-juncs —no-novel-indels —raw-juncs. The —mate-inner-dist and —mate-std-dev arguments were calculated using the MISO exon_utils.py script, which maps reads to constitutively spliced exon junctions.

Step 4: The reads aligned to splice junctions were filtered as in step 2.

Step 5: All resulting BAM files were merged to create a combined file of all aligned RNA-seq reads.

Identification and Quantification of Differential Splicing

Isoform ratios for all alternative splicing events were quantified using MISO v2.0. Constitutively spliced exons and introns were quantified using junction-spanning reads. The conditional knockin and knockout mice were compared in a pair-wise manner, and for each pair the analysis was restricted to splicing events with 20 or more reads supporting either or both isoforms, and where the event was alternatively spliced in the sample pair. From that subsets of events, those that fulfilled the following criteria were defined as differentially spliced: (i) they had at least 20 relevant reads in both samples, (ii) the change in absolute isoform ratio was ≥10%, and (iii) the statistical analysis of isoform ratios had a Bayes factor greater than or equal to 5, when calculated using Wagenmakers's framework. The human AML samples were analyzed by calculating the median isoform ratios across all 28 SRSF2 wildtype samples, and comparing that in a pair-wise manner to each SRSF2 mutant sample, using the same methodology as for the knockin and knockout mice. The Mx1-Cre⁺Srsf2^{+/+} or Mx1-Cre⁺Srsf2^{P95H/+}E7107-treated mice were compared in a pair-wise manner against the median isoform ratios of their vehicle-treated counterparts, using the same methodology. For the MLL-AF9 AML transformed mice there were sufficient replicates to do a group-based comparison within the Srsf2P95H and wildtype genotypes individually (N=5 for each genotype-treatment combination). E7107- and vehicle-treated mice were compared in a two-sided Wilcoxon rank-sum test, using the total number of isoform reads within each treatment group. Events were categorized as being differentially spliced if they fulfilled the following criteria: (i) they had at least 20 relevant reads in both samples, (ii) the change in median absolute isoform ratio was ≥10%, and (iii) they had a P-value<0.01.

### Example 1

### Therapeutic Targeting of Cells Exhibiting Spliceosome Machinery Mutations (SMM)

The present inventors have examined new means to alter splicing fidelity prior to disease onset. To this end, the following approaches were identified as means to target splicing which exhibit preferential effects in SMM leukemias.

Reducing Splicing Fidelity Through Inhibition of Protein Arginine Methyltransferases (PRMTs):

It has been reported that inhibiting spliceosomal assembly through inhibition of protein arginine methylation of Sm proteins provides an alternate means of therapeutic splicing inhibition [Koh, C. M. et al., Nature (2015) 523: 96-100]. Specifically, genetic deletion or pharmacologic inhibition of PRMT5, reduced symmetric dimethylation of Sm proteins, a process required for small nuclear RiboNucleoProtein (snRNP) and spliceosome-assembly [Koh, C. M. et al. Nature (2015), supra]. Based on these results, the present inventors have now discovered that reducing splicing fidelity by inhibiting arginine methylation results in strong preferential killing of SF-mutant leukemia cells over their wildtype counterparts.

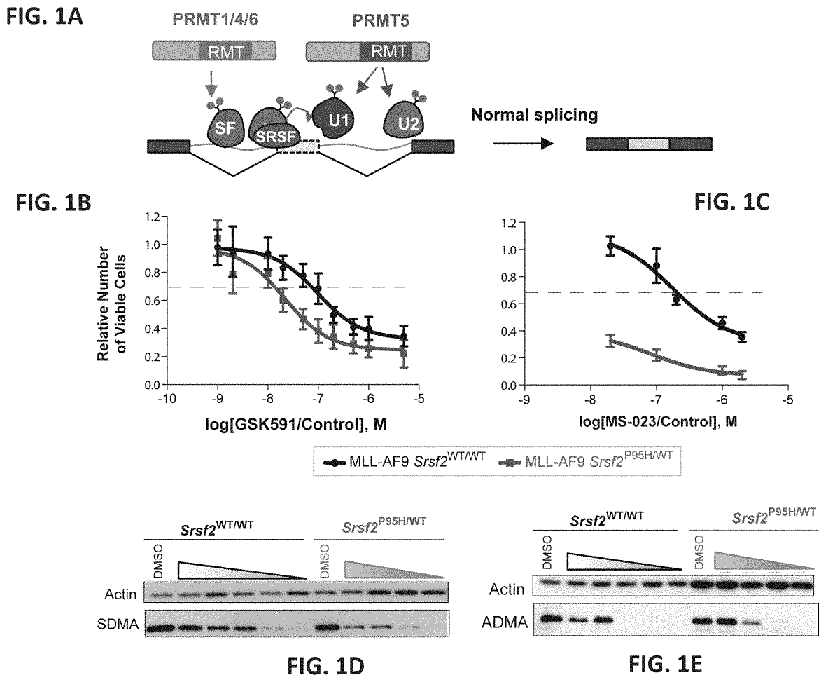

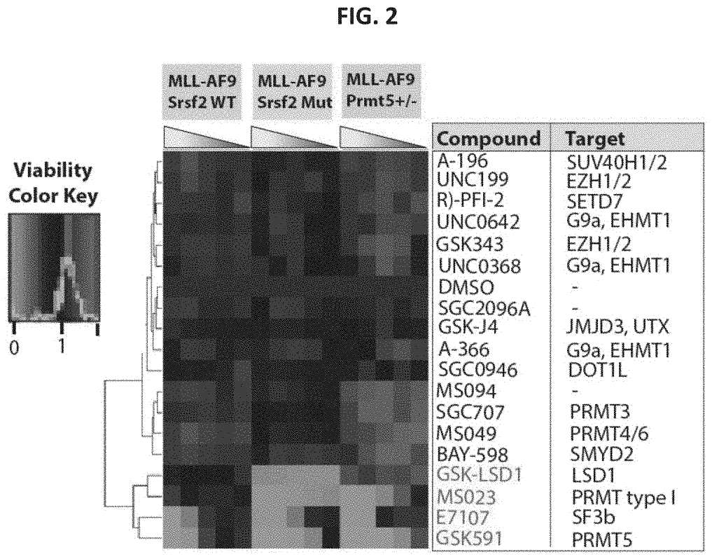

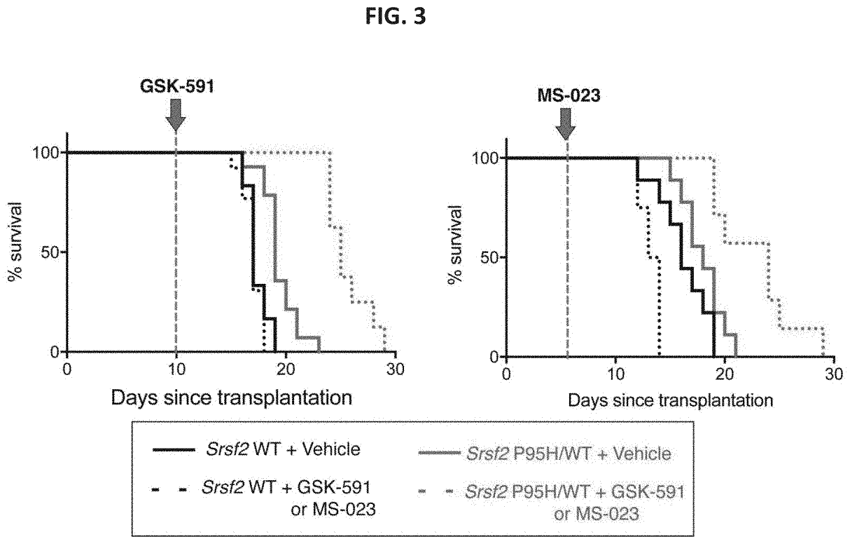

These studies revealed that Srsf2-mutant cells were more sensitive to the PRMT5 inhibitor (GSK591) and the pan-PRMT type I inhibitor (MS-023) (FIGS. 1A-E). Of note, the SF3B1 inhibitor, E7107, was used in the same assays as a positive control. Similar mutant selective effects were seen in in vivo testing of GSK591 and MS-023 (FIG. 3). Of note, both Type I (PRMT1, PRMT4 and PRMT6) as well as type II PRMT enzymes (PRMT5 and PRMT9), which catalyze asymmetric and symmetric dimethylation of arginines respectively, methylate components of the spliceosome. As shown in FIG. 2, AML cells with SMMs or partial loss of PRMT5 to inhibitors of splicing, PRMTs, or LSD1. MLL-AF9 Srsf2^{WT/WT}; MLL-AF9 Srsf2^{P95H/WT} or MLL-AF9 Prmt5^{+/-} cells were treated for 7 days with the indicated compounds in 384 wells (5 increasing concentrations per compound). Viability at day 7 was scored by MTS assay and reported as the ratio over control treated cells (with equivalent dilution of DMSO). The experiment was conducted in biological triplicate, and each individual run was repeated in technical triplicate.

Perturbing Splicing Through Proteasomal Degradation of RBM39:

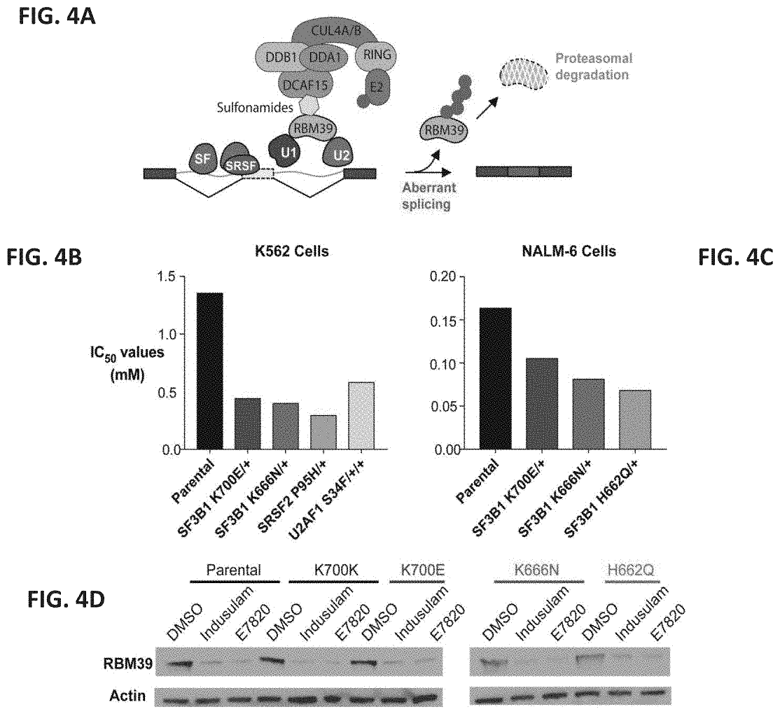

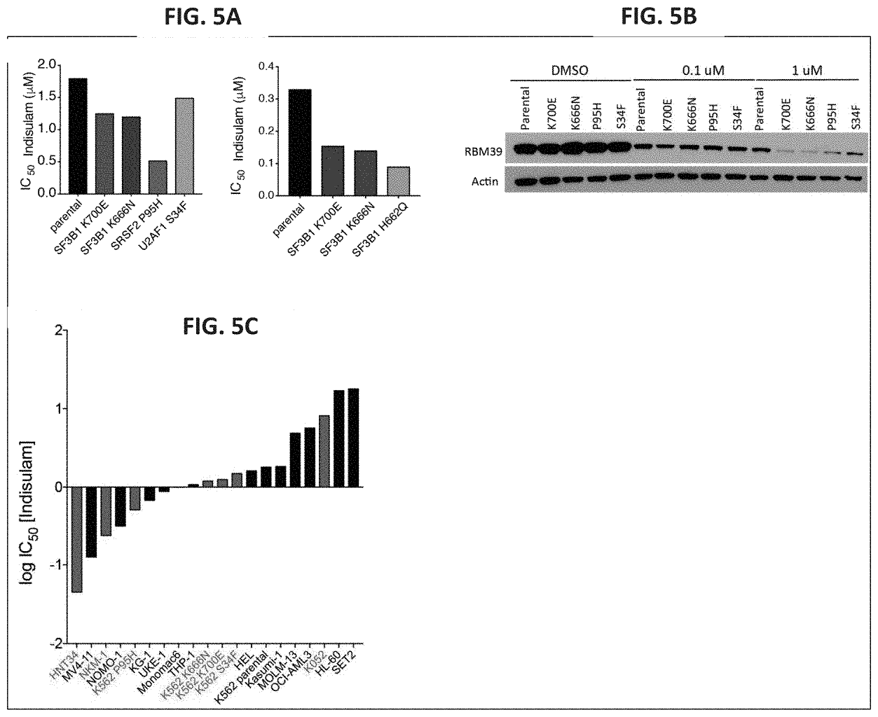

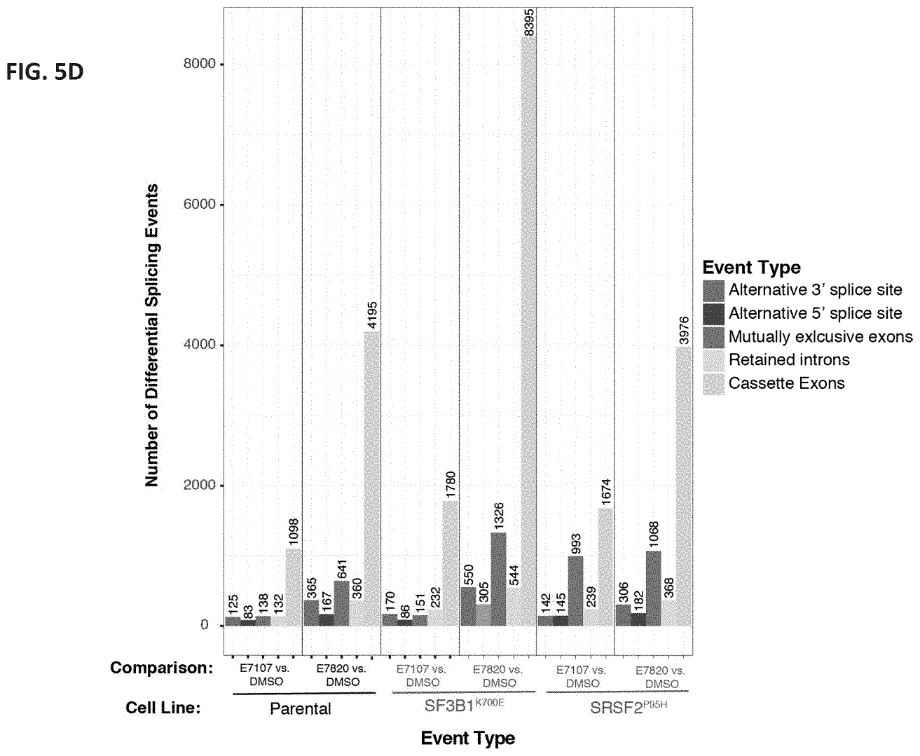

It was previously discovered that a class of sulfonamide drugs with known anti-cancer properties inhibit splicing as their dominant mechanism of anti-tumor action [Uehara, T. et al., Nat Chem Biol (2017) 13: 675-680]. Interestingly, these drugs utilize the cellular ubiquitin ligase machinery to promote proteosomal degradation of the key spliceosomal protein RBM39. Based on these observations, the present inventors have identified that sulfonamides exhibit preferential effects on cells bearing SMMs (FIGS. 4A-D). This includes data demonstrating preferential effects of indisulam on isogenic cells with or without mutations in RNA splicing factors as well as on AML cells with naturally occurring mutations in RNA splicing factors (FIGS. 4A-D and FIGS. 5A-C). These experiments were performed using both indisulam and the sulfonamide analogue E7820.

The effects of indisulam and the sulfonamide analogue, E7820, were first examined across a panel of 18 genetically diverse AML cell lines. Measurement of cell viability upon sulfonamide exposure revealed broad anti-leukemic effects with potent inhibitory activity across many AML subtypes, with most cell lines exhibiting sub-micromolar sensitivity. It was found that leukemia cells bearing mutations in leukemia-associated mutations in splicing factors were amongst the most sensitive cells to sulfonamides. In addition, a number of AML cell lines without spliceosomal gene mutations also exhibited sensitivity to the sulfonamides. Evaluation of relative DCAF15 mRNA expression revealed that the highest and lowest relative levels of DCAF15 mRNA correlated with greatest and worst response to E7820 respectively. The preferential effects of sulfonamides on leukemia cells bearing spliceosomal gene mutations was further confirmed in a series of isogenic AML lines (K562 and TF-1) engineered to express hotspot mutations in SF3B1, SRSF2, and U2AF1 from their endogenous loci and B-cell acute lymphoblastic leukemia (NALM-6 cells) with exposure to E7820 and indisulam. In each instance, spliceosomal mutant cells were preferentially sensitive to growth inhibition to sulfonamides over spliceosomal wild-type cells. In isogenic cells, E7820 exposure led to similar dose-dependent degradation of RBM39 in leukemia cell lines.

To understand the basis for the preferential effects of sulfonamides on spliceosomal mutant cells, RBM39 protein levels across a panel of isogenic AML cells—with or without knockin of spliceosomal gene mutations—were evaluated. This showed that degradation of RBM39 occurred in a comparable dose-dependent fashion across cell lines regardless of spliceosomal gene mutation status (FIG. 5B), suggesting that a greater dependency on residual wild-type splicing function may be the basis for increased sensitivity of spliceosomal mutant cells to sulfonamides. Given that spliceosomal mutant cells are preferentially sensitive to alterations in splicing over their wild-type counterparts, RNA-sequencing was performed of parental K562 and isogenic lines expressing SF3B1^{K700E} and SRSF2^{P95H} mutations treated with 1 μM of E7820 (which represents the IC₅₀ of parental K562 cells to E7820; FIG. 5D). In parallel, RNA-seq was carried out of the same cell lines treated with E7107, a small molecule that inhibits splicing through impeding binding of SF3B1 to the branch point [Finci et al., Genes & Dev. (2018) 32: 309-320]. Treatment with either E7820 or E7107 at the IC₅₀ of each drug in parental K562 cells resulted in increased cassette exon skipping and intron retention relative to DMSO treatment regardless of spliceosomal gene mutation status. Interestingly, however, at equipotent non-toxic doses, E7820 resulted in greater changes in splicing within each cell type and across each category of splicing versus E7107 at this dose. Moreover, a greater number of differential splicing events were identified within each type of splicing in SF3B1^{K700E} cells treated with E7820 versus SF3B1 wild-type counterparts. These data suggest that at least one reason for the preferential effects of sulfonamides on SF3B1-mutant over wild-type cells is a difference in splicing response of SF3B1-mutant cells to RBM39 degradation.

The present inventors also noticed that a number of differentially spliced events upon sulfonamide exposure involved mRNAs encoding RNA binding proteins (RBPs) upregulated and required for AML cell survival. These included SUPT6H, hnRNPH, and SRSF10, where E7820 exposure resulted in intron retention that was most pronounced in spliceosomal mutant cells. In addition, RBM39 degradation also resulted in enhanced aberrant splicing of the HOXA9 target genes BMI-1 and MYB and a number RBPs in spliceosomal mutant AML over WT counterparts, including aberrant splicing events in U2AF2 and RBM3. Moreover, gene set enrichment analysis of differentially spliced events in response to E7820 also revealed downregulation of targets of MYC and PI3K-AKT-mTOR signaling as well as mRNAs involved in response to inflammation all known to be important in AML pathogenesis or progression. Thus, both genetic as well pharmacologic degradation of RBM39 disrupts splicing as well as other pathways required for leukemogenicity. Overall, these data suggest that the presence of spliceosomal gene mutations as well as DCAF15 expression may serve as important predictors of response to RBM39 degradation in AML.

### Example 2

### Therapeutic Targeting of SMM Cells Before AML/MDS Diagnosis

Each of the above therapeutic approaches has been evaluated in the context of overt MDS, AML, and related myeloid malignancies, and has demonstrated preferential effects on cells carrying SMMs. The present inventors are testing the effect splice inhibitors, e.g. PRMT inhibitors and sulfonamides, in the pre-leukemic phase and in preclinical models of ARCH as well as in vitro and in vivo on preL-HSPCs carrying SMMs.

The Efficacy of Spliceosome Modulatory Compounds on Pre-AML Bearing SMMs In Vitro:

To determine responsiveness of preL-HSPCs to spliceosome modulatory compounds, the present inventors are co-culturing CD34⁺ preL-HSPCs carrying SMMs together with MS5 cells. The drugs being tested include clinical-grade SF3b modulatory compound (H3B-8800), a Type II PRMT inhibitor (GSK3326595), a Type I PRMT inhibitor (MS-023), a sulfonamide compound (E7820), or vehicle control treatment. 10,000 preL-HSPCs per well (6 well plate) are cultured for 72 hours before adding the drugs for another 72 hours. Cells post exposure undergo FACS analysis and are analyzed for allelic burden of SMMs as well as splicing and gene expression by RNA-seq. These in vitro tests allow to better understand the mechanisms of cell death and survival under the various therapies, and to test different drug combinations.

Study the Efficacy of Spliceosome Modulatory Compounds on Pre-AML Bearing SMMs In Vivo:

Samples capable of engrafting both myeloid and lymphoid cells (multi-lineage xenografting) which carry SMMs following engraftment are used in these studies, together with the auto-transplant bags from AML patients at remission capable of multi-lineage xenografting. To test whether any treatment modulating SMMs is selectively targeting the preL-HSPCs, the total human engraftment is measured in treated and untreated control groups. In addition, VAF of SMMs in human cells extracted from mice, are measured in treated versus non-treated mice. CD34⁺ cells (10,000-50,000) are injected intrafemorally (IF) to 5 treated mice and 5 untreated mice. On day 35 post-injection mice are treated with the following compounds: H3B-8800, GSK3326595, MS-023, E7820, or vehicle control. On day 56 following cells injection, mice are sacrificed. Other control experiments include the same experiments, using CD34⁺ cells derived from human cord blood and from elderly people with no recurrent pre-leukemic mutations. To provide evidence that targeting preL-HSPCs is more favorable than treating full-blown leukemia, the present inventors are also injecting all the AML samples with SMMs to NSG and SGM3 mice and identifying leukemic engraftment (CD33⁺). After leukemic engrafting samples are identified, they are injected and analyzed again now with therapy versus vehicle as proposed above for preL-HSPCs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of preventing a hematopoietic disorder or malignancy in a subject at risk of developing the hematopoietic disorder or malignancy due to one or more mutation in a splicing factor, wherein the subject has not been diagnosed as having the hematopoietic disorder or malignancy, and wherein the subject is positive for said one or more mutation in said splicing factor, the method comprising administering to said subject an agent capable of inhibiting spliceosomal activity, with the proviso that said agent does not inhibit RBM39 activity.

2. The method of claim 1, wherein said agent capable of inhibiting spliceosomal activity is an agent capable of inhibiting a protein arginine methyltransferase (PRMT) as set forth in EC numbers 2.1.1.319, 2.1.1.320 or 2.1.1.321.

3. The method of claim 1, wherein said agent capable of inhibiting spliceosomal activity is a splicing inhibitor.

4. The method of claim 1, wherein said agent capable of inhibiting spliceosomal activity is a proteasomal degradation compound.

5. The method of claim 2, wherein the PRMT is selected from the group consisting of a protein arginine methyltransferase 1 (PRMT1), a protein arginine methyltransferase 3 (PRMT3), a protein arginine methyltransferase 4 (PRMT4), a protein arginine methyl transferase 5 (PRMT5), a protein arginine methyltransferase 6 (PRMT6) and a protein arginine methyltransferase 9 (PRMT9).

6. The method of claim 2, wherein said agent capable of inhibiting said PRMT is a polypeptide, a polynucleotide, or a small molecule.

7. The method of claim 2, wherein said agent is a type I PRMT inhibitor MS-023 dihydrochloride, or a derivative or analog thereof.

8. The method of claim 5, wherein when said PRMT comprises:
PRMT5, said agent comprises GSK591 dihydrochloride or GSK3326595, or a derivative or analog thereof; or
PRMT1, said agent comprises C-21, Furamidine dihydrochloride or TC-E 5003, or a derivative or analog thereof; or
PRMT3, said agent comprises SGC707 or UNC2327, or a derivative or analog thereof; or
PRMT4, said agent comprises MS049 oxalate salt or TP064, or a derivative or analog thereof; or
PRMT6, said agent comprises MS049 oxalate salt, or a derivative or analog thereof.

9. The method of claim 3, wherein said splicing inhibitor is a polypeptide, a polynucleotide, or a small molecule.

10. The method of claim 3, wherein said splicing inhibitor is selected from the group consisting of a Sudemycin, a Spliceostatin, a FR901464, a Pladienolide, a Herboxidiene, a Meayamycin, an Isoginkgetin, a Madrasin, a Tetrocarcin, a N-palmitoyl-L-leucine, a Psoromic acid, a Clotrimazole, a NSC635326, a Napthazarin, an Erythromycin, a SAHA, a Garcinol, an Okadaic acid, a NB-506, a Ubistatin, a G5, or a derivative or analog thereof.

11. The method of claim 3, wherein said splicing inhibitor is selected from the group consisting of a E7107, H3B-8800, FD-895, GEX1Q1-5, RQN-18690, NSC659999, BN82865, NSC95397, tetracycline, streptomycin, splitomicin, tautomycin, microcystin, siospyrin, chlorhexidine, or a derivative or analog thereof.

12. The method of claim 4, wherein said proteasomal degradation compound targets a spliceosome associated protein selected from the group consisting of a core member of the SF3b complex, a U2AF complex, or a PRMT enzyme and a RNA binding protein.

13. The method of claim 12, wherein said proteasomal degradation compound targets a spliceosome associated protein selected from the group consisting of SF3B1, SF3B2, SF3B3, PHF5a, U2AF1, U2AF2, PRMT5, PRMT1, PRMT2, PRMT3, PRMT4, PRMT6, PRMT8, SUPT6H, hnRNPH, and SRSF10.

14. The method of claim 1, wherein said mutation is in a splicing factor selected from the group consisting of U2AF1, SF3B1, SRSF2, and ZRSR2.

15. The method of claim 1, wherein said mutation is a point mutation.

16. The method of claim 14, wherein said mutation is a mutation in S34 or Q157 in said U2AF1 polypeptide.

17. The method of claim 14, wherein said mutation is a R625L, a N626H, a K700E, a G740E, a K741N, a Q903R, a E622D, a R625G, a Q659R, a H662Q, a H662D, a K666Q, a K666E, a K666N, a K666T, a K666R or a G742D mutation in said SF3B1 polypeptide.

18. The method of claim 14, wherein said mutation is a mutation in P95 in said SRSF2 polypeptide.

19. The method of claim 1, wherein said mutation is detected in pre-leukemic hematopoietic stem and progenitor cells.

20. The method of claim 1, wherein the hematopoietic disorder or malignancy is a leukemia or a myelodysplastic syndrome (MDS).
